SEQUENCE LISTING (26 sequences)

SEQ ID NO: 1 - protein, 40 aa, Heterocephalus glaber
LDNLQNLVHMLASSKGCPLPQTRGPELLENLGGALKESIY

SEQ ID NO: 2 - protein, 39 aa, Cavia porcellus
LENLQSLVQMMASSKGCPLPHTTEPELQDLESTLKESRY

SEQ ID NO: 3 - protein, 40 aa, Homo sapiens
LENLRDLLHVLAFSKSCHLPWASGLETLDSLGGVLEASGY

SEQ ID NO: 4 - protein, 40 aa, Pan troglodytes; identical to SEQ ID NO: 3

SEQ ID NO: 5 - protein, 40 aa, Macaca fascicularis
LENLRDLLHLLAFSKSCHLPLASGLETLESLGDVLEASLY

SEQ ID NO: 6 - protein, 40 aa, Mus musculus
LENLRDLLHLLAFSKSCSLPQTSGLQKPESLDGVLEASLY

SEQ ID NO: 7 - protein, 40 aa, Gallus gallus; identical to SEQ ID NO: 6

SEQ ID NO: 8 - protein, 40 aa, Rattus rattus
LENLRDLLHLLAFSKSCSLPQTRGLQKPESLDGVLEASLY

SEQ ID NO: 9 - protein, 40 aa, Cricetulus griseus
LENLRDLLHLLASSKSCSLPPTSELQQLESLDGVLEASLY

SEQ ID NO: 10 - protein, 40 aa, Ovis aries
LENLRDLLHLLAASKSCPLPQVRALESLESLGVVLEASLY

SEQ ID NO: 11 - protein, 40 aa, Bos taurus; identical to SEQ ID NO: 10

SEQ ID NO: 12 - protein, 40 aa, Sus scrofa
LENLRDLLHLLASSKSCPLPQARALETLESLGGVLEASLY

SEQ ID NO: 13 - protein, 40 aa, Canis lupus
LENLRDLLHLLASSKSCPLPRARGLETFESLGGVLEASLY

SEQ ID NO: 14 - protein, 7 aa, Artificial Sequence
  Consnesus sequence
  MISC_FEATURE (1)..(1): X is G or S
  MISC_FEATURE (3)..(3): X is S, H or P
  MISC_FEATURE (6)..(6): X is Q, H, W, L , P, or R
  MISC_FEATURE (7)..(7): X is T, A, or V
XCXLPXX

SEQ ID NO: 15 - protein, 167 aa, Homo sapiens
MHWGTLCGFLWLWPYLFYVQAVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 16 - protein, 6 aa, Mus musculus
SCSLPQ

SEQ ID NO: 17 - protein, 6 aa, Mus musculus
CSLPQT

SEQ ID NO: 18 - protein, 6 aa, Mus musculus
SLPQTS

SEQ ID NO: 19 - protein, 6 aa, Mus musculus
PQTSGL

SEQ ID NO: 20 - protein, 6 aa, Mus musculus
GLQKPE

SEQ ID NO: 21 - protein, 6 aa, Mus musculus
LQKPES

SEQ ID NO: 22 - protein, 10 aa, Homo sapiens
SCHLPWASGL

SEQ ID NO: 23 - protein, 15 aa, Mus musculus
SCSLPQTSGLQKPES

SEQ ID NO: 24 - protein, 9 aa, Homo sapiens
CHLPWASGL

SEQ ID NO: 25 - protein, 6 aa, Homo sapiens
SCHLPW

SEQ ID NO: 26 - protein, 6 aa, Homo sapiens
CHLPWA

(19) United States
(12) Patent Application Publication
(10) Pub. No.: US 2020/0255492 A1
(43) Pub. Date: Aug. 13, 2020

(54) LEPTIN PEPTIDES AND THEIR USE FOR TREATING NEUROLOGICAL DISORDERS

(71) Applicants: UNIVERSITY OF DUNDEE, Dundee (GB); UNIVERSITY COURT OF THE UNIVERSITY OF ST ANDREWS, St Andrews, Fife (GB)

(72) Inventors: Jenni HARVEY, Dundee (GB); Gayle DOHERTY, St Andrews, Fife (GB)

(21) Appl. No.: 16/642,627

(22) PCT Filed: Aug. 30, 2018
(86) PCT No.: PCT/GB2018/052441

§ 371 Date: Feb. 27, 2020

Related U.S. Application Data
(60) Provisional application No. 62/553,050, filed on Aug. 31, 2017.

(51) Int. Cl. C07K 14/575; A61P 25/28

(52) U.S. Cl. CPC C07K 14/5759; A61K 38/00; A61P 25/28

(57) ABSTRACT

A method of treating a neurological disorder comprising administering a leptin peptide fragment comprising amino acids located within the region of amino acids 116-125 of leptin is disclosed. The leptin peptide fragment preferably comprises up to 30 amino acids, and/or wherein the leptin peptide fragment comprises one or more amino acids located between amino acids 116-122 of leptin, for example the sequence X₁CX₂LPX₃X₄ wherein X₁ is selected from G or S; X₂ is selected from S, H or P; X₃ is selected from Q, H, W, L, P or R and X4 is selected from T, A, or V (SEQ ID NO:14) or the sequence SCHLPWASGL (SEQ ID NO:22). The neurological disorder can include those which would benefit from treatment through cognitive enhancement and/or neuroprotection, such as age-associated memory impairment or loss, mild cognitive impairment, and Alzheimer's disease, and can include Parkinson's disease, frontotemporal dementia, progressive supranuclear palsy, Pick's disease, corticobasal degeneration, alcoholic dementia, (DLB) dementia with Lewy bodies, Picks' disease, thalamic dementia, hippocampal sclerosis, Hallervorden-Spatz, multiple system atrophy, tauopathies, subacute aterioscleroitic encephalopathy (Binswanger's disease), amyloid angiopathy, vasculitis, prion diseases, and paraneoplastic syndromes. The invention also includes a pharmaceutical formulation for this method, which can include the peptide in the form of a cyclic peptide or a peptide conjugate.

### FIELD OF THE INVENTION

The present invention relates to leptin peptide fragments and methods of using such peptide fragments in treating or protecting from neurological conditions, including use in cognitive enhancement and/or neuroprotection.

### BACKGROUND TO THE INVENTION

Alzheimer's disease (AD) is a complex, progressive brain disorder that results in profound cognitive deficits, particularly in memory. Accumulation of toxic amyloid plaques and neurofibrillary tangles comprising hyper-phosphorylated tau are key pathological features of AD. Proteolytic processing of APP and generation of toxic amyloid beta (Aβ) is linked to aberrant synaptic function and neuronal degeneration.

Clinical evidence indicates that diet and lifestyle are major risk factors for developing AD and disruption to metabolic systems is linked to AD (Stranahan and Mattson 2012). The metabolic hormone leptin regulates energy homeostasis, but also markedly influences hippocampal synaptic function. Indeed, leptin-insensitive rodents exhibit impaired hippocampal LTP and spatial memory (Li et al. 2002). Moreover, direct administration of leptin into the hippocampus facilitates learning (Wayner et al. 2004), whereas hippocampal dendritic morphology, glutamate receptor trafficking and synaptic plasticity are significantly altered after leptin treatment (Shanley et al. 2001; O'Malley et al. 2007; Moult et al. 2010; Luo et al. 2015). Clinical studies indicate that aberrant leptin function is associated with an increased risk of AD, as AD patients exhibit significantly lower leptin levels than normal (Power et al. 2001). Individuals with lower circulating leptin also have a greater risk of developing AD (Lieb et al. 2009). Additionally, leptin levels are markedly reduced in rodent models with familial AD mutations (APPSwe; PSIM146V; Fewlass et al. 2004). In cellular models of AD, leptin treatment prevents the aberrant effects of Aβ, as leptin reverses the ability of Aβ to inhibit LTP and facilitate LTD in hippocampal slices (Doherty et al. 2013) and it prevents Aβ-driven internalization of the AMPA receptor subunit, GluA1 in hippocampal neurons (Doherty et al. 2013). Chronic intra-cerebroventricular injection of leptin also reverses Aβ-induced impairments in LTP in vivo (Tong et al. 2015). In cell survival assays, leptin protects against Aβ-induced toxicity as neuronal viability is increased after leptin treatment. Moreover the cortical expression of 2 AD-linked biomarkers, endophilin 1 and phosphorylated tau, are enhanced in leptin-insensitive Zucker fa/fa rats (Doherty et al. 2013). Thus there is growing evidence that leptin-based therapies may be beneficial in AD.

However, using leptin therapeutically may not be the best approach due to its widespread central actions. One possibility is to develop small molecules that mimic leptin action. Indeed, studies have found that specific fragments of the leptin peptide are bioactive and mirror the anti-obesity effects of leptin (Grasso et al. 1997; Rozhayskaya-Arena et al. 2000). Indeed, application of a C-terminal fragment of leptin (amino acids 116-130) or a shorter fragment (116-121) to leptin-deficient ob/ob mice reduced food intake and body weight (Grasso et al. 1997; Rozhayskaya-Arena et al. 2000; Grasso et al. 2001). However, another leptin fragment (22-56) is also bioactive as significant reductions in food intake have been observed after leptin (22-56) administration (Samson et al. 1996). Thus, it is feasible that different parts of the leptin molecule are active in the CNS. However, the cognitive enhancing and neuroprotective properties of the bioactive leptin fragments are unknown.

### SUMMARY OF THE INVENTION

In a first aspect there is provided a leptin peptide fragment or variant thereof for use in a method of treating or preventing development of a neurological disorder.

The leptin peptide fragment of the present invention may be up to 30, 25, 20, 15, or 10 amino acids in length and comprises amino acids located within the region of amino acids 116-125 of leptin (see the table below adapted from Peptides. 2012 December; 38(2); 326-336). Typically the leptin peptide is at least 4, 5, 6 or 7 amino acids in length.

| | Sequence | SEQ |
|---|---|---|
| Name | 101 111 121 131 | ID NO |
| NAKED_MOLE_RAT | LDNLQNLVHM LASSKGCPLP QTRGPELLEN LGGALKESIY | 1 |
| GUINEA_PIG | LENLQSLVQM MASSKGCPLP HTTEPELQD- LESTLKESRY | 2 |
| HUMAN | LENLRDLLHV LAFSKSCHLP WASGLETLDS LGGVLEASGY | 3 |
| COMMON_CHIMPANZEE | LENLRDLLHV LAFSKSCHLP WASGLETLDS LGGVLEASGY | 4 |
| CRAB_EATING_MACAQUE | LENLRDLLHL LAFSKSCHLP LASGLETLES LGDVLEASLY | 5 |
| MOUSE | LENLRDLLHL LAFSKSCSLP QTSGLQKPES LDGVLEASLY | 6 |
| CHICKEN | LENLRDLLHL LAFSKSCSLP QTSGLQKPES LDGVLEASLY | 7 |
| RAT | LENLRDLLHL LAFSKSCSLP QTRGLQKPES LDGVLEASLY | 8 |
| CHINESE_HAMSTER | LENLRDLLHL LASSKSCSLP PTSELQQLES LDGVLEASLY | 9 |
| SHEEP | LENLRDLLHL LAASKSCPLP QVRALESLES LGVVLEASLY | 10 |
| CATTLE | LENLRDLLHL LAASKSCPLP QVRALESLES LGVVLEASLY | 11 |
| PIG | LENLRDLLHL LASSKSCPLP QARALETLES LGGVLEASLY | 12 |
| DOG | LENLRDLLHL LASSKSCPLP RARGLETFES LGGVLEASLY | 13 |

As can be seen from the above, leptin sequences (using the conventional 1-letter amino acid code employed in the table and used throughout herein) are highly conserved in the region which the present invention is directed to.

In one embodiment, the leptin peptide fragment includes sequences located between amino acids 116-122. Based on the above, a consensus sequence for amino acids 116-122 may be as follows:

X₁CX₂LPX₃X₄

wherein X₁ is selected from G or S; X₂ is selected from S, H or P; X₃ is selected from Q, H, W, L, P or R and X₄ is selected from T, A, or V (SEQ ID NO: 14).

Conveniently the peptide sequence may comprise, consist essentially of, or consist of amino acids 116-121, 117-122, 117-125, 118-123, 119-124, 120-125, or 116-130.

In certain embodiments the invention is directed to human therapies and so the leptin peptide fragment may be based on the human sequence. The human leptin sequence and numbering is shown below, with amino acids 116-125 highlighted:

```
(SEQ ID NO: 15)
                10 20 30 40
MHWGTLCGFL WLWPYLFYVQ AVPIQKVQDD TKTLIKTIVT
                50 60 70 80
RINDISHTQS VSSKQKVTGL DFIPGLHPIL TLSKMDQTLA

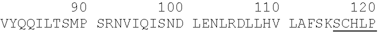

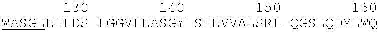


LDLSPGC
```

In certain embodiments the neurological disorder is a disorder which would benefit from treatment through cognitive enhancement and/or neuroprotection.

The term “neurological disorder” refers to disorders of the nervous system that result in impairment of neuronal mediated functions and includes disorders of the central nervous system (e.g., the brain, spinal cord) as well as the peripheral nervous system.

The invention relates to leptin peptide fragments for use in methods of treating acute, chronic and prophylactic treatment of neurologic and neurodegenerative diseases, attenuation of acute or chronic neuronal damage in neurological disease (“neuroprotection”), and prophylaxis of neurological diseases.

The leptin peptides fragments may be useful for enhancing cognitive function and/or synaptic plasticity in vivo, e.g., for the treatment and/or prevention of memory impairment in mammalian subjects such as humans. In particular, the leptin peptide fragments may be useful for the treatment and/or prevention of age-associated memory impairment or loss, mild cognitive impairment, and Alzheimer's disease. In one embodiment the leptin peptide fragments may be useful for short term enhancement of cognitive function and/or synaptic plasticity. Cognitive enhancement may in addition include enhancement of synaptic plasticity.

“Cognition” generally refers to the process of obtaining, organizing, and using knowledge. Enhancing cognitive function refers to enhancing any aspect of this process, e.g., learning, the performance of mental operations, the storage and/or retrieval of information or thoughts (memory), and/or preventing a decline from a subject's current state. Numerous standardized tests can be used to evaluate cognitive function. Such tests can be used to identify subjects in need of enhancement of cognitive function and/or to monitor the effects of treatment. Suitable tests include, but are not limited to, the Mini-Mental Status Exam (Folstein, 1975), components of the PROSPER neuropsychological test battery (Houx, 2002), etc. Family history, age, and other factors may also be used to identify subjects in need of enhancement of cognitive function.

“Synaptic plasticity” is defined as the ability of a synapse to change its strength in response to a pattern of stimulation (i.e., one or more electrical or chemical stimuli), wherein the alteration in strength typically outlasts the event that triggers it. A synapse that exhibits this property is said to be plastic, or to display synaptic plasticity. A neural network in which some or all of the synapses exhibit plasticity is also said to exhibit synaptic plasticity. Synaptic plasticity may be considered to exist at the level of the presynaptic terminal, the postsynaptic terminal, or both. Thus a synapse is said to exhibit presynaptic plasticity if presynaptic strength is altered in response to a pattern of stimulation. A synapse is said to exhibit postsynaptic plasticity if postsynaptic strength is altered in response to a pattern of stimulation, and/or if the probability that an action potential will be generated in response to a second pattern of stimulation is altered as a result of a first pattern of stimulation.

“Synaptic strength” of a given synapse may be assessed by measuring one or more indicators of presynaptic strength, postsynaptic strength, or both. In general, presynaptic strength refers to properties including (i) the amount of neurotransmitter released in response to a pattern of stimulation; and/or (ii) the probability of neurotransmitter release in response to a pattern of stimulation. The product of (i) and (ii) provides an overall measure of presynaptic strength. Postsynaptic strength refers to properties including (i) the size of the postsynaptic current or potential induced by a fixed amount of neurotransmitter or other stimulus, e.g., an electrical stimulus; and/or (ii) the probability of firing of an action potential for a fixed amount of input. Overall synaptic strength reflects a combination of presynaptic and postsynaptic strength. Overall synaptic strength may be determined by combining measures of presynaptic and postsynaptic strength (e.g., by adding, multiplying, etc.). Alternatively, overall synaptic strength may be measured directly, e.g., by stimulating individual presynaptic neuron(s) and recording the evoked response at the corresponding postsynaptic neuron(s). For purposes of the present invention, a synapse will be said to increase its synaptic strength if it increases its presynaptic strength or its postsynaptic strength, or both. A synapse will be said to decrease its synaptic strength if it decreases its presynaptic strength or its postsynaptic strength, or both. One of ordinary skill in the art will recognize that other parameters indicative of synaptic strength may be used, and parameters may be combined in various ways to arrive at a measurement of synaptic strength. One of ordinary skill in the art will also appreciate that a variety of measurement techniques may be applied to assess parameters associated with synaptic strength.

Synaptic plasticity is believed to be essential for the processes involved in learning and memory. Thus compositions that enhance synaptic plasticity are of use for the treatment of individuals (subjects) suffering from any of a variety of conditions in which cognitive function, e.g., memory and/or learning is impaired. The compositions are also useful to prevent the onset of such conditions. These conditions include, but are not limited to, those known as “benign senescent forgetfulness”, “age-associated memory impairment”, “age-associated cognitive decline”, “mild cognitive impairment”, Alzheimer's disease, dementias (associated with any of a number of causes), attention-deficit disorder, etc. The compositions and methods of the invention may also find use to enhance the cognitive function, e.g., memory and/or learning capacity of normal individuals, i.e., individuals not suffering from any clinically recognized condition or disorder. They may be useful on a short-term basis or may be administered chronically.

AD may be diagnosed according to the National Institute of Neurological and Communicative Disorders and Stroke—Alzheimer's Disease and Related Disorders Association criteria for a clinical diagnosis of probable Alzheimer's disease, imaging and various biomarkers (e.g., levels of tau protein in cerebrospinal fluid). In addition, individuals with dominant mutations in the amyloid precursor protein, PS1, or PS2 genes are at increased risk of AD. It has also been found that the risk of developing AD is greater in individuals with the ε4 allele of the gene encoding ApoE. Such individuals may be particularly appropriate candidates for therapy with the compositions described herein.

The term “neuroprotection” refers to prevention or a slowing in neuronal degeneration, including, for example, neuronal death and/or axonal loss.

The term “Subject”, as used herein, refers to an individual to whom a leptin peptide fragment is to be delivered. Preferred subjects are mammals, particularly domesticated mammals (e.g., dogs, cats etc.), primates, or humans. The subject may be a human being, e.g., a human being suffering from or at risk of a neurological disease or condition such as age-associated memory loss, mild cognitive impairment, or Alzheimer's disease. Typically the subject will be administered a leptin peptide fragment comprising a sequence which is based on the subjects endogenous leptin sequence. Thus, a human may be administered a leptin peptide fragment comprising sequence based on the human leptin sequence. However, this should not be construed as being essential.

Mild cognitive impairment (MCI) refers to the transitional zone or time period between normal aging and mild dementia. Criteria for the diagnosis of MCI may include subjective and objective memory impairment, normal cognitive and activities of daily living (ADL), and the absence of any specific criteria for dementia. The cognitive impairment may be amnestic (memory) or involve any other isolated cognitive domain that is greater than expected for normal aging. The patient and family may have insight into the impairment, but the patient is still able to function adequately with ADL. The objective memory function detected by neuro-psychological tests usually 1.5 SD below the average performance of individuals with similar age and education. MRI of the brain may reveal mild atrophy of the hippocampus and entorhinal cortex while neuropathologic studies can reveal some early features of dementia. Thus, while subjects with MCI have a condition that differs from normal aging and are likely to progress to dementia at an accelerated rate, not all patients progress to dementia. Finally, most subjects with MCI that convert to dementia have elevated levels of CSF tau protein.

Cognitive decline may occur in various other neurological diseases which have dementia as a symptom and which may have either a genetic predisposition (chromosome 17), contain Lewy bodies or tau proteins. For example, mutations of tau occur in families with FTDP-17 (frontal temporal dementia linked with Parkinson's disease). This syndrome is characterized by widespread NFT formation associated with tau, in the absence of amyloid deposits. Thus, abnormalities of tau structure and function produces progressive, severe neuronal degeneration and death. Additional dementing illnesses include Parkinson's disease, frontotemporal dementia, progressive supranuclear palsy, Pick's disease, corticobasal degeneration, alcoholic dementia, (DLB) dementia with Lewy bodies, Picks' disease, thalamic dementia, hippocampal sclerosis, Hallervorden-Spatz, multiple system atrophy, tauopathies, subacute aterioscleroitic encephalopathy (Binswanger's disease), amyloid angiopathy, vasculitis, prion diseases, and paraneoplastic syndromes. Those skilled in the art will recognize that these diseases are not Alzheimer's disease or an MCI condition, but may be treated in accordance with the present invention.

As well as the leptin peptide fragments identified herein, the present invention extends to variants thereof. Variants include peptides with one or more substitutions, deletions and/or additions. Variants also include chemical modifications to the N-terminus, C-terminus and/or backbone amino acids, which do not substantially negatively alter the activity of the peptide. By substantially negatively alter the activity of the peptide means that the activity is not reduced by more than 10%, 5%, or 1% as compared the unmodified peptide. Of course such chemical modifications could have a positive effect on activity and any modifications which have a positive effect on activity are included.

A variant of the present invention includes a variant of a parent leptin peptide fragment having at least about 80%, 90% identity and most preferably at least about 95% identity to the parent molecule and which has cognitive enhancing and/or neuroprotective activity. In a preferred embodiment, the variant peptide has an amino acid sequence which differs by 3, 2 or 1 amino acid(s), from the leptin peptide fragments identified herein.

In one embodiment, the leptin peptide fragment variant comprises between one and three amino acid deletions (or additions) from the leptin peptide fragments identified herein, providing that the variant peptide still has cognitive enhancing and/or neuroprotective activity. Based on the teaching herein, the skilled addressee can easily test such variant peptides in order to determine whether or not they possess or are likely to possess cognitive enhancing and/or neuroprotective activity and hence be useful in accordance with the present invention. It is also possible through comparison with unmodified leptin peptide fragments to determine whether or not a modified peptide has an altered activity and by how much. Suitable examples of tests are described herein.

Variants also include peptide conjugates. Peptide conjugates may generally include a further biologically active agent being conjugated to the leptin peptide fragment, such as through the N or C-terminal amino acid of the leptin peptide fragment. The other biologically active agent may be a further peptide, for example, which may facilitate translocation of the leptin peptide fragment across the gut and/or blood brain barrier and/or may itself have cognitive enhancing and/or neuroprotective properties. A further envisaged conjugate may be the leptin peptide conjugated to itself, that is two leptide peptides of the present invention conjugated to another, by appropriate means.

Peptides composed of L-amino acids undergo rapid proteolysis in the gut, making oral administration, the method generally associated with the highest patient compliance, often problematic. Additionally, peptides degrade fairly rapidly in serum and therefore must be administered in large doses which often can cause numerous adverse side effects and serious toxicity. As peptides are expensive to manufacture, high dosage levels contribute significantly to the overall cost of peptide therapeutics. Furthermore, the flexibility of the peptide structure in solution is often associated with low biological activity and/or selectivity. Thus, it may be appropriate to modify the peptides of the present invention in order to address and/or obviate the above problems.

Some post-translational modification strategies have found use in peptide engineering The simplest and most commonly used approach is to include N-terminal acetylation and C-terminal amidation. Natural peptides may be halogenated, such as brominated or occasionally chlorinated, and it may be desired to modify the peptides of the present invention by halogenation of particular residues, such as tryptophan residues. Enhanced peptide stability may result from increase in size or hydrophobicity due to halogenation, or protect the peptide from degradation/oxidation. Incorporation of D-amino acids provides another useful approach for improvement of peptide stability. Alternatively, partial incorporation of D-amino acids may also improve peptide stability.

Another modification known to the skilled addressee, is to cyclize peptides. Following the pioneering work of R. Schwyzer [Ludecher, U., et al., Helv. Chim. Acta 54. 1637 (1971)] on gramicidin S, conformational restriction of peptides by medium and long range cyclization has been extensively employed. In addition to other modes of conformational restriction, such as configurational and structural alteration of amino acids, local backbone modifications, short-range cyclization etc., medium and long range cyclization [Hruby, V. J., Life Sci. 31, 189 (1982); Kessler, H., Angew. Chem. Int. Ed. Eng., 21, 512 (1982); Schiller, P. W., in the “Peptides”, Udenfriend, S., and Meienhofer, J. Eds., Volume 6 p. 254 (1984); Veber, D. F. and Freidinger, R. M., Trends in Neurosci. 8, 392 (1985); Milner-White, E. J., Trends in Pharm. Sci. 10, 70 (1989)] is used for the following purposes: biologically active peptides are cyclized to achieve metabolic stability, to increase potency, to confer or improve receptor selectivity and to control bioavailability. The possibility of controlling these important pharmacological characteristics through cyclization of linear peptides prompted the use of medium and long range cyclization to convert natural bioactive peptides into peptidomimetic drugs. Cyclization also brings about structural constraints that enhance conformational homogeneity and facilitate conformational analysis [Kessler, H., Angew. Chem. Int. Ed. Eng., 21, 512 (1982)]. Moreover, the combination of structural rigidification-activity relationship studies and conformational analysis gives insight into the biologically active conformation of linear peptides.

Conformationally restricted peptides containing medium and long range cyclizations have been mainly prepared following the same modes of cyclization of homodetic and heterodetic natural peptides. These include: a side-chain to side-chain cyclization (usually the formation of a lactam ring and/or an —S—S— bond through cyclization of functional groups already present in the native sequence or by substitution of other amino acids with Glu and Lys or Cys respectively); b end to end cyclization (previously called backbone to backbone cyclization [Manesis, N. J. and Goodman, M., Org. Chem., 52, 5331 (1987)]) and c side-chain to end groups cyclization.

Another mode of cyclization includes side-chain to amino end and side-chain to carboxyl end. The exact location, type and size of the ring (which can also be controlled by “spacers” [Manesis, N. J. and Goodman, M., Org. Chem., 52, 5331 (1987)]) to achieve maximum selectivity and activity is determined mainly by Structure-Activity-Relationship (SAR) considerations in conjunction with conformational analysis.

In another embodiment the peptides of the present invention may be cyclized by use of an enzyme cleavable linker. In this manner, the N-terminal amino group and the C-terminal carboxyl group of the peptide is linked via a linker, or the C-terminal carboxyl group of the peptide is linked to a side chain amino group or a side chain hydroxyl group via a linker, or the N-terminal amino group of said peptide is linked to a side chain carboxyl group via a linker, or a side chain carboxyl group of said peptide is linked to a side chain amino group or a side chain hydroxyl group via a linker. Useful linkers include 3-(2′-hydroxy-4′,6′-dimethyl phenyl)-3,3-dimethyl propionic acid linkers and its derivatives and acyloxyalkoxy derivatives linkers—see for example U.S. Pat. No. 5,672,584.

The present invention, therefore, can be extended to include cyclizing the peptide of the invention with a compound (i.e. a “linker”) which is (a) capable of being reacted with the peptide in a cyclizing reaction scheme to produce a cyclic peptide and optionally (b) capable of re-linearizing the peptide by means of in vivo enzymes to linearize the peptide.

Other methods of cyclizing the peptides of the present invention include the methods described in WO2014001822 and WO2016071422 to which the skilled reader is directed and the entire contents of which are hereby incorporated herein by way of reference.

Thus, in a further aspect there is provided a cyclic form of a peptide as described herein. There is also provided pharmaceutical formulations comprising such cyclic peptides, as well as their use in method of treating the diseases and conditions discussed herein.

The invention further provides a method of treating a neurological disorder in a subject, such as a disorder which would benefit from treatment through cognitive enhancement and/or neuroprotection, comprising administering to the subject an effective amount of a leptin peptide fragment as described herein.

An “effective amount” of a leptin peptide fragment refers to the amount of leptin peptide fragment which is sufficient to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. A desired biological response may be, for example, (i) an increase in synaptic plasticity; (ii) an improvement in a task requiring cognitive function, e.g., improved performance on a test that measures learning and/or memory; (iii) a slowing in the rate of decline in cognitive function (e.g. neuroprotection), e.g., as measured by performance on a test that measures learning and/or memory. An effective amount in humans may be less than 10 □m, such as less than 100 nm.

Different dosing regiments may likewise be administered, typically at the discretion of the medical practitioner. As the leptin peptide fragments are based on a natural molecule (i.e leptin) it is expected that the fragments are likely to display low toxicity and allow for at least daily administration although regimes where the compound(s) is (or are) administered more infrequently, e.g. every other day, weekly or fortnightly, for example, are also embraced by the present invention.

“Treating”, when used with respect to a desired therapeutic effect in a subject such as a human being, can include reversing, alleviating, inhibiting the progress of, preventing, or reducing the likelihood of the disease, disorder, or condition to which such term applies, or one or more symptoms or manifestations of such disease, disorder or condition. “Preventing” refers to causing a disease, disorder, condition, or symptom or manifestation of such, or worsening of the severity of such, not to occur.

The leptin peptide fragments of the invention may be administered at intervals during the time over which treatment is required or is deemed necessary. For example, the leptin peptide fragments can be administered 3-4 times daily, 1-2 times daily, every other day, weekly, etc. It may be preferred to maintain an effective concentration within the body over a time period during which treatment is desired. Since, in general, it is desirable to maintain cognitive function throughout life, the compounds may be administered indefinitely.

The peptides of this invention can exist as stereoisomers or mixtures of stereoisomers; for example, the amino acids which comprise them can have the configuration L-, D-, or be racemic independently of each other. Therefore, it is possible to obtain isomeric mixtures as well as racemic mixtures or diastereomeric mixtures, or pure diastereomers or enantiomers, depending on the number of asymmetric carbons and on which isomers or isomeric mixtures are present. The preferred structures of the peptides of the invention are pure isomers, i.e., enantiomers or diastereomers.

For example, when it is stated that an amino acid, can be —S—, it is understood that the amino acid, is selected from —L—S—, —D—S— or mixtures of both, racemic or non-racemic. The preparation and processes described in this document enable the person skilled in the art to obtain each of the stereoisomers of the peptide of the invention by choosing the amino acid with the right configuration.

In the context of this invention, the term “amino acids” includes the natural amino acids codified by the genetic code as well as non-codified amino acids, whether they are natural or not. Examples of non-codified amino acids are, without restriction, citrulline, ornithine, sarcosine, desmosine, norvaline, 4-aminobutyric acid, 2-aminobutyric acid, 2-aminoisobutyric acid, 6-aminohexanoyc acid, 1-naphthylalanine, 2-naphthylalanine, 2-aminobenzoic acid, 4-aminobenzoic acid, 4-chlorophenylalanine, 2,3-diaminopropionic acid, 2,4-diaminobutyric acid, cycloserine, carnitine, cystine, penicillamine, pyroglutamic acid, thienylalanine, hydroxyproline, allo-isoleucine, allo-threonine, isonipecotic acid, isoserine, phenylglycine, statin, B-alanine, norleucine, N-methyl amino acids, a-amino acids and [3-amino acids, among others, as well as their derivatives. A list of unnatural amino acids can be found in the article “Unusual amino acids in peptide synthesis” by D. C. Roberts and F. Vellaccio, in The Peptides, Vol. 5 (1983), Chapter VI, Gross E. and Meienhofer J., Eds., Academic Press, New York, USA or in the commercial catalogues of the companies specialized in the field.

Synthesis of the peptides of the invention, their stereoisomers, mixtures thereof and/or their pharmaceutically acceptable salts can be carried out according to conventional methods, known in the prior art, such as using solid phase peptide synthesis methods [Stewart J. M. and Young J. D., “Solid Phase Peptide Synthesis, 2nd edition”, (1984), Pierce Chemical Company, Rockford, Ill.; Bodanzsky M. and Bodanzsky A., “The practice of Peptide Synthesis”, (1994), Springer Verlag, Berlin; Lloyd-Williams P. et a “Chemical Approaches to the Synthesis of Peptides and Proteins”, (1997), CRC, Boca Raton, Fla., USA], synthesis in solution, a combination of the methods of solid phase synthesis and synthesis in solution or enzymatic synthesis [Kullmann W. “Proteases as catalysts for enzymic syntheses of opioid peptides”, (1980), J. Biol. Chem., 255(17), 8234-8238].

The peptides can also be obtained by fermentation of a bacterial strain, modified or unmodified, by genetic engineering to produce the desired sequences, or by controlled hydrolysis of the full length leptin molecule obtained from a suitable animal or human source, to release the leptin peptide fragments of the invention.

There is further provided an in vitro method of preparing a leptin peptide fragment as described herein, or modified form thereof.

For use in methods according to the present invention, the leptin peptide fragments or a physiologically acceptable salt, solvate, ester or amide thereof described herein may be presented as a pharmaceutical formulation, comprising the leptin peptide fragment or physiologically acceptable salt, ester or other physiologically functional derivative thereof, together with one or more pharmaceutically acceptable carriers therefor and optionally other therapeutic and/or prophylactic ingredients. Any carrier(s) are acceptable in the sense of being compatible with the other ingredients of the formulation and not deleterious to the recipient thereof.

Examples of physiologically acceptable salts of the compounds according to the invention include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

The determination of physiologically acceptable esters or amides, particularly esters is well within the skills of those skilled in the art.

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc., depending on the number of water molecules present per molecule of substrate.

It will be appreciated that the compounds of the present invention may exist in various stereoisomeric forms and the compounds of the present invention as hereinbefore defined include all stereoisomeric forms and mixtures thereof, including enantiomers and racemic mixtures. The present invention includes within its scope the use of any such stereoisomeric form or mixture of stereoisomers, including the individual enantiomers of the compounds of formulae (I) or (II) as well as wholly or partially racemic mixtures of such enantiomers.

Pharmaceutical formulations include those suitable for oral, topical (including dermal, buccal and sublingual), rectal or parenteral (including subcutaneous, intradermal, intramuscular and intravenous), nasal and pulmonary administration e.g., by inhalation. The formulation may, where appropriate, be conveniently presented in discrete dosage units and may be prepared by any of the methods well known in the art of pharmacy. Methods typically include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged, e.g., in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms, e.g., tablets wherein an active compound is formulated in an appropriate release-controlling matrix, or is coated with a suitable release-controlling film. Such formulations may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles.

Injectable preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active leptin peptide fragment may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range of 0.5 to 7 microns are delivered in the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 100 microlitres, upon each operation thereof.

As a further possibility an active compound may be in the form of a solution or suspension for use in an atomizer or nebuliser whereby an accelerated airstream or ultrasonic agitation is employed to produce a fine droplet mist for inhalation.

Formulations suitable for nasal administration include preparations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particle diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, an appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Pharmaceutically acceptable carriers are well known to those skilled in the art and include, but are not limited to, 0.1 M and preferably 0.05 M phosphate buffer or 0.8% saline. Additionally, pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, anti-oxidants, chelating agents, inert gases and the like.

Formulations suitable for topical formulation may be provided for example as gels, creams or ointments. Such preparations may be applied e.g. to a wound or ulcer either directly spread upon the surface of the wound or ulcer or carried on a suitable support such as a bandage, gauze, mesh or the like which may be applied to and over the area to be treated.

Liquid or powder formulations may also be provided which can be sprayed or sprinkled directly onto the site to be treated, e.g. a wound or ulcer. Alternatively, a carrier such as a bandage, gauze, mesh or the like can be sprayed or sprinkle with the formulation and then applied to the site to be treated.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w and preferably 60 to 80% w/w of the active ingredient(s) and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain the compound or a derivative or salt thereof and may optionally include a veterinarily acceptable water-miscible solvent, for example polyethylene glycol, propylene glycol, glycerol, glycerol formal or such a solvent mixed with up to 30% v/v of ethanol. The liquid concentrates may be administered to the drinking water of animals.

### BRIEF DESCRIPTION OF THE DRAWINGS

The present invention will now be further described by way of example, with reference to the following figures.

FIG. 1A, FIG. 1B, and FIG. 1C are graphs of normalized iEPSP slope versus time.

FIG. 2A, FIG. 2B, FIG. 2C, FIG. 2D, FIG. 2E, and FIG. 2F are graphs of normalized slope (% baseline) versus time (FIG. 2A and FIG. 2B); a photograph of representative confocal images of surface GluR1 staining in control cultured hippocampal neurons and after exposure to leptin (FIG. 2C; a bar graph of relative intensity for Control, Leptin, Leptin (116-130) and Leptin 22-56); and a bar graph of relative intensity versus Control, Leptin, BpV, Bpv plus Leptin, Leptin 116 and BpV plus Leptin 116.

FIG. 3A, FIG. 3B, FIG. 3C and FIG. 3D are graphs of normalized fEPSP slope versus time.

FIG. 4A, FIG. 4B, FIG. 4C, and FIG. 4D are graphs of normalized fEPSP slope versus time (FIG. 4A, FIG. 4B, FIG. 4C and FIG. 4D) and 4E shows a bar graph of relative intensity for Aβ (41-1), Aβ, Leptin, Leptin plus Aβ, Leptin 116 (116-130 peptide), Leptin 116 plus Aβ, Leptin 22 (22-56 peptide) and Leptin 22 plus Aβ.

FIG. 5A, FIG. 5B, FIG. 5C and FIG. 5D are bar graphs showing the percent LDH release for human Leptin and Leptin (116-130); CuCl2-treated (FIG. 5A), Aβ1-41-treated (FIG. 5B), and untreated (FIG. 5C).

FIG. 6A, FIG. 6B and FIG. 6C are bar graphs showing the present LDH release for Aβ1-42, Leptin (116-130), WP1066, Leptin (116-130) plus wortmannin, and wortmannin (FIG. 6A); the ratio of p-STAT3:pan-STAT3 for untreated or Leptin (116-130) (FIG. 6B); and the ratio of p-Akt:pan-Akt for untreated and Leptin (116-130) (FIG. 6C).

FIG. 7A, FIG. 7B, FIG. 7C and FIG. 7D are a drawing showing object-place-context recognition (FIG. 7A); and bar graphs showing dimension index (FIG. 7B), total exploration time (FIG. 7C) and exploration time (FIG. 7D) for control, leptin, and fragment.

FIGS. 8A and 8C are graphs of normalised fEPSP slope over time for leptin 116-121 and leptin 124-129 respectively; FIGS. 8B and 8D are bar graphs of relative intensity vs control for leptins 116-121, 117-122 and leptins 124-129, 125-130; FIG. 8E is a bar graph of relative intensity above control for leptin 116-121, 117-122, 118-123, 120-125 and 124-129.

FIG. 9A and FIG. 9B are representative confocal images of surface GluA1 expression in hippocampal neurons and a bar graph showing relative intensity of surface GluA1 for Control and the three indicated Leptin fragments. FIG. 9c is a bar graph of relative intensity above control for leptin 116-121, 117-122, 118-123, 120-125, 124-129 and 125-130.

FIG. 10 is a diagram showing the sequences and activities of Leptin fragments. Leptin (116-121) (SCSLPQ, SEQ ID NO:16); Leptin (117-122) (CSLPQT, SEQ ID NO:17); Leptin (118-123) (SLPQTS, SEQ ID NO:18); Leptin (120-125) (PQTSGL, SEQ ID NO:19); Leptin (124-129) (GLQKPE, SEQ ID NO:20); Leptin (116-130) (SCSLPQTSGLQKPES, SEQ ID NO:21). Leptin (125-130) (LQKPES, SEQ ID NO:22)

FIG. 11A, FIG. 11B, and FIG. 11C are three bar graphs showing % LDH release, % mitochondrial activity, and ptau expression, respectively, for the indicated Leptin fragments.

FIG. 12: Aβ_{1_42} promotes increased expression of tau at dendrites. A) Representative confocal images of endogenous tau labelling in control (HBS) and Aβ_{1_42} (1 μM) treated hippocampal (7-14 days in vitro) neurons, with magnified views of dendritic regions indicated by white boxes and intensity profiles of corresponding dendrites. Aβ_{1_42} increased endogenous tau expression compared to control. Scale bars are 10 gm. B) Pooled data showing relative tau intensity in control and Aβ_{1_42}-treated neurons. Aβ_{1_42} promotes tau mislocalization to dendrites. C) Representative images of endogenous tau staining in control conditions and after exposure to the inactive, reverse peptide of Aβ_{1_42} (Aβ_{42_1}; 1 μM) and Aβ_{1_42} (1 μM). Aβ_{42_1} has no effect on the endogenous levels of tau compared to control whereas Aβ_{1_42} increases dendritic levels of tau. D) Pooled data showing relative tau intensities for control, Aβ_{42_1} and Aβ1-42- ** * represent p<0.001.

FIG. 13: Aβ₁₋₄₂ promotes increased expression of tau at synapses. A) Representative confocal images of endogenous tau (red) and PSD-95 (green) labelling in control (HBS), Aβ₄₂₋₁ (1 μM) and Aβ₁₋₄₂ (1 μM) treated hippocampal (7-14 DIV) neurons, with magnified dendritic regions indicated by white boxes and intensity profiles of corresponding dendrites. Aβ₁₋₄₂ increases % co-localization between endogenous tau and PSD-95 compared to Aβ₄₂₋₁ and control-treated neurons. Scale bars are 10 ium. B) Pooled data showing % co-localization for control, Aβ₄₂₋₁ and Aβ₁₋₄₂ treated neurons. Aβ₁₋₄₂ promotes tau mislocalization to dendritic spines. C) Pooled data showing relative tau intensity for control, Aβ₄₂₋₁ and Aβ₁₋₄₂.

FIG. 14: Leptin prevents Aβ-induced mislocalization of tau to synapses. A. Representative confocal images of endogenous tau (red) and PSD-95 (green) labelling in control (HBS), leptin (10 nM), Aβ1-42 (1 μM) and leptin (10 nM)+Aβ1-42 (1 μM) treated hippocampal neurons. Leptin reduces the levels of tau and its expression at synapses. B) Pooled data showing % co-localization for control, leptin, Aβ1-42 and Aβ1-42+leptin-treated neurons. Leptin prevented the Aβ1.42-driven movement of tau to synapses. C) Pooled data showing relative tau intensity in control, leptin, Aβ1-42 and Aβ1-42+leptin-treated neurons. Leptin counteracts Aβ1-42's effects by reducing the dendritic levels of tau to near control levels.

FIG. 15: Leptin(116-130) prevents AP-induced mislocalization of tau to synapses. A) Representative confocal images of endogenous tau (red) and PSD-95 (green) labelling in control (HBS), leptin(116-130; 10 nM), Aβ1_42 (1 μM) and leptin(116-130; 10 nM)+Aβ1-42 (1 μM) treated neurons. Leptin(116-130) reduces the dendritic levels of tau and its synapses. Scale bars=B) Pooled data showing % co-localization for control, leptin(116-130), Aβ1-42 and Aβ1-42+leptin(116-130)-treated neurons. Leptin(116-130) prevented Aβ1-42-driven movement of tau to synapses. C) Pooled data showing relative tau intensity in control, leptin(116-130), Aβ1_42 and Aβ1-42+leptin(116-130)-treated neurons. Leptin(116-130) counteracts Aβ1-42's effects by reducing dendritic tau levels.

FIG. 16. Treatment with Aβ₁₋₄₂ increases phosphorylation of tau. A: Confocal images of phosphorylated tau (p-Tau) staining in control treated (HBS), Aβ₁₋₄₂ (1 μM) treated, and Aβ₄₂₋₁ (1 μM) treated hippocampal cultures. Scale bar represents 10 μm. B: Histogram of pooled data from changes in p-Tau localization in control conditions, and after exposure to Aβ₁₋₄₂, or Aβ₄₂₋₁ in hippocampal neurons. Dashed line denotes changes compared to control intensity. *, **, and *** represent p<0.05, p<0.01, and p<0.001, respectively.

FIG. 17. The synaptic levels of p-Tau are increased by Aβ1-42. A. Confocal images of p-Tau (red) and PSD-95 (green) staining in control (HBS), Aβ1.42 (1 μM) and Aβ42.1 (1 μM) treated cultures. (B) Histogram of pooled data showing changes in p-Tau localization in control conditions, and after exposure to Aβ1.42, or Aβ42.1 in hippocampal neurons (n=4). (C) Histogram of pooled data showing p-tau/PSD-95 co-localisation in control conditions, and after exposure to Aβ1.42, or Aβ42-1 in hippocampal neurons (n=3). (D) Histogram of pooled data of % co-localization of p-Tau and PSD-95 (n=3). Dashed line denotes changes compared to control intensity

FIG. 18. Leptin protects against Aβ₁₋₄₂ effects on p-tau. (A). Confocal images of p-Tau staining in control (HBS), Aβ₁₋₄₂ (1 μM), leptin (10 nM), and Aβ₁₋₄₂ (1 μM)+leptin (10 nM) treated cultures. (B) Histogram of pooled data showing changes in p-Tau localization in control conditions, and after exposure to Aβ₁₋₄₂, leptin and Aβ₁₋₄₂+leptin. C: Histogram of pooled data of % co-localization of pTau and PSD-95 in neurons exposed to Aβ₁₋₄₂, leptin or Aβ₁₋₄₂+leptin. Leptin prevents Aβ₁₋₄₂-driven tau phosphorylation and its targeting to synapses.

FIG. 19. The leptin fragment (116-130) also protects against the effects of Aβ₁₋₄₂ A. Histogram of pooled data illustrating changes in p-Tau localization in control conditions, and after exposure to Aβ₁₋₄₂, leptin frag (116-130) and Aβ₁₋₄₂±leptin (116130) in hippocampal neurons (n=2). B: Histogram of pooled data of the % co-localization of p-Tau and PSD-95 after exposure to Aβ₁₋₄₂, leptin(116-130) and Aβ₁₋₄₂+leptin(116-130) in hippocampal neurons. (n=2). Treatment with leptin(116-130) prevents Aβ₁₋₄₂-dependent phosphorylation of tau and it trafficking to synapses.

FIG. 20: Leptin prevents Aβ-induced mislocalization of tau by inhibiting GSK-3β. A) Representative confocal images of tau (red) and PSD-95 (green) labelling in Aβ1-42 (1 μM)+leptin (10 nM), Aβ1-42 (1 μM)+SB216763 (100 nM) and Aβ1-42 (1 μM)+SB216763 (100 nM)+leptin (10 nM) treated hippocampal neurons. Inhibition of GSK-3β reduces tau expression at synapses. B) Pooled data showing % co-localization for tau and PSD95 under various conditions. Inhibition of GSK-3β mirrored the effects of leptin as it decreased % tau/PSD-95 co-localization. C) Pooled data showing relative tau intensity in neurons under various conditions. Inhibition of GSK-3β by SB216763 mirrors the effects of leptin suggesting that the protective actions of leptin are due to inhibition of GSK-3β.

FIG. 21: A: Bar chart of the quantification of the fluorescent signal per cell following thioflavin S staining of SH-SY5Y neuronal cultures 96 hours after seeding with 1 μM Amyloid (Aβ₁₋₄₂). Cultures were established in triplicate on 3 separate occasions and statistical significance relative to control untreated cultures is denoted by *** where P<0.001. Also shown are fluorescent photomicrographs (B) of thioflavin S-stained control untreated cultures, cultures treated with 1 nM leptin₁₁₆₋₁₃₀, 1 μM Ab₁₋₄₂ or co-treated with 1 nM leptin₁₁₆₋₁₃₀ and 1 μM Ab₁₋₄₂(B). Thioflavin S is used to stain for amyloid in these cultures and it is clear that amyloid propagation following seeding is greatly reduced in the presence of leptin₁₁₆₋₁₃₀. Scale bar represents 10 μm.

FIG. 22: Bar graph (A) demonstrating the percent survival of SH-SY5Y neural cells following serum/glucose deprivation (an emerging in vitro model of stroke [2]). Cultures were established on 7 separate occasions in quadruplicate and starved of serum and glucose when they reached 70% confluence. Neural cell viability in response to a range of concentrations of leptin₁₁₆₋₁₃₀ was determined 96 hours later using a crystal violet assay to measure cell number. Statistical significance relative to serum/glucose deprivation is denoted with ** where P<0.01 and *** where P<0.001. Photomicrographs of serum/glucose deprived cultures and of serum/glucose deprived cultures treated with 0.1 nM leptin₁₁₆₋₁₃₀ (B) further highlight the enhanced cell number in the presence of leptin₁₁₆₋₁₃₀. Scale bar represents 25 μm.

FIG. 23: Bar graph demonstrating an increase in the cross-sectional area of individual mitochondria following 1 hour of treatment with 100 μM 6-OHDA. In combination with 0.1 nM leptin₁₁₆₋₁₃₀(A), leptin₁₁₆₋₁₃₀ prevents 6-OHDA-associated mitochondrial swelling. Also shown is a bar graph demonstrating the decrease in mitochondrial fragmentation following treatment with 100 μM 6-OHDA. In combination with 0.1 nM leptin₁₁₆₋₁₃₀ (B), leptin₁₁₆₋₁₃₀ prevents 6-OHDA-associated mitochondrial clumping. Cultures were established on 3 separate occasions in quadruplicate and stained with MitoRED to identify mitochondria after 1 hour of treatment. Statistical significance relative to untreated control is denoted with ** where P<0.01 and *** where P<0.001. Representative images of 100 μM 6-OHDA-treated MitoRED stained cultures (C) and 100 μM 6-OHDA and leptin₁₁₆₋₁₃₀ co-treated MitoRED stained cultures are shown. Scale bar represents 10 μM.

FIG. 24. Bar chart of the quantification of the fluorescent signal per cell following thioflavin S staining of neuronal cultures 96 hours after seeding with 1 μM Amyloid (Ab₁₋₄₂). Cultures were established in triplicate on 3 separate occasions and statistical significance relative to control untreated cultures is denoted by *** where P<0.001 (A). Bar chart demonstrating the discrimination index of exploration time of novel, compared to familiar, objects in a murine episodic-like memory test (B). 3 groups of mice (n=8 per group) were IP injected with saline (control) on 7.8 nM/ml leptin₁₁₆₋₁₂₁ or leptin₁₁₇₋₁₂₁. Memory performance was tested 45 minutes post-injection. Statistical significance relative to saline-injected animals is denoted by * where P<0.05. Bar chart of serum leptin levels in mice injected with leptin₁₁₆₋₁₂₁ or leptin₁₁₇₋₁₂₂ demonstrating that administration of these hexamers did not significantly alter the level of native leptin (C). Leptin levels were determined by ELISA, using a commercially-supplied kit (Sigma, UK) and using blood serum harvested 24 hours post injection.

FIG. 25. Alignment of murine and human target sequences within the flexible C-D loop region of the leptin molecule (A). Bar chart showing the quantification of the fluorescent signal per cell following thioflavin S staining of neuronal cultures 96 hours after seeding with 1 μM Amyloid (Ab₁₋₄₂) (B). Cultures were established in triplicate on 3 separate occasions and statistical significance relative to control untreated cultures is denoted by *** where P<0.001.

FIG. 26. Bar graphs demonstrating the survival of fully differentiated SH-SY5Y human neuronal cells following administration of either Aβ₁₋₄₂ (10 μM; A, B) or 10 μM CuCl₂ (C,D). Data from crystal violet assays to determine cell number (A, C) or lactate dehydrogenase assays to quantify the degree of cell membrane rupture (B, D) are shown, Cultures were established on 3 separate occasions in triplicate and viability was determined 96 hours post treatment. Statistical significance relative to control, untreated cells is denoted with * where P<0.05; ** where P<0.01 and *** where P<0.001.

### DETAILED DESCRIPTION OF EMBODIMENTS OF THE INVENTION

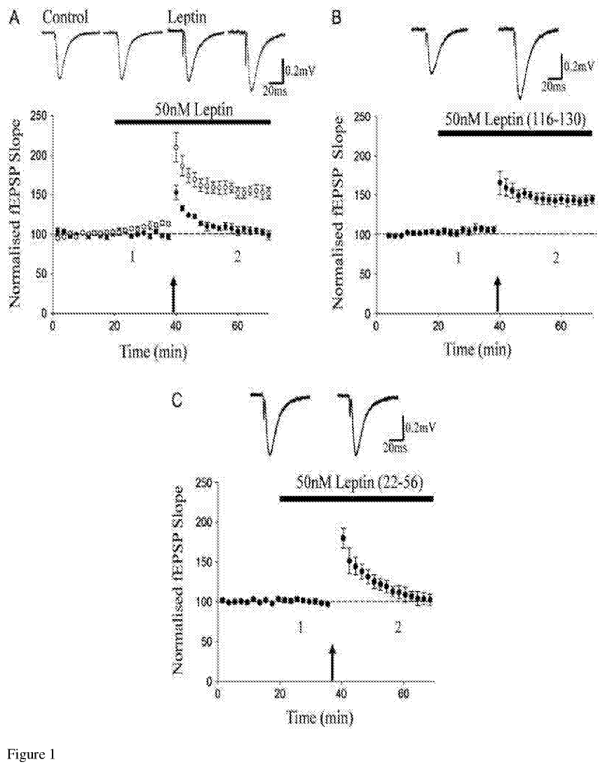

FIG. 1A, FIG. 1B, and FIG. 1C show that Leptin (116-130) promotes conversion of STP into a persistent increase in synaptic transmission in juvenile hippocampus. FIG. 1A: pooled data showing that primed burst stimulation (indicated by the arrow) delivered in the absence of leptin induced STP (filled circle) in juvenile hippocampal slices. In contrast, in leptin-treated slices (open circle) the same stimulation paradigm resulted in a persistent increase in synaptic transmission. In this and subsequent figures, each point is the average of 4 successive responses. Top, Representative synaptic records (average of 4 consecutive records) are shown for the times indicated. FIG. 1B and FIG. 1C: during exposure to leptin (116-130; 50 nM), application of the primed burst stimulation paradigm resulted in a persistent increase in synaptic transmission FIG. 1B, whereas only STP was evident in slices treated with leptin (22-56; FIG. 1C);

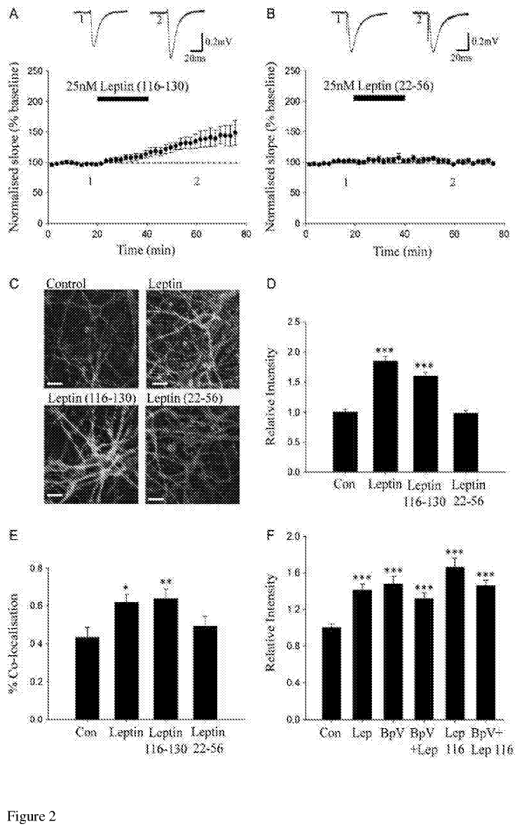

FIG. 2A, FIG. 2B, FIG. 2C, FIG. 2D, FIG. 2E, and FIG. 2F show that in adults, leptin (116-130) induces a persistent increase in synaptic efficacy and increases GluA1 trafficking to hippocampal synapses. FIG. 2A: pooled data showing that application of leptin (116-130) results in a persistent increase in excitatory synaptic transmission in adult hippocampal slices. FIG. 2B: in contrast application of leptin (22-56) failed to alter excitatory synaptic strength. FIG. 2C: representative confocal images of surface GluR1 staining in control cultured hippocampal neurons and after exposure to leptin, leptin (116-130) or leptin (22-56). Leptin (116-130) mirrors the effects of leptin by increasing GluA1 surface labelling. Scale bars, 10 μm. FIG. 2D: pooled data showing relative changes in surface GluA1 labelling in control conditions, and after exposure to leptin, leptin (116-130) or leptin (22-56) in hippocampal neurons. FIG. 2E: pooled data of the percent colocalization of surface GluA1 and synaptophysin immunolabelling in cultured hippocampal neurons. Leptin (116-130), but not leptin (22-56) increased GluA1 surface labelling associated with synapses. FIG. 2F: pooled data of relative changes in surface GluA1 labelling in hippocampal cultures in control conditions, after bpV, leptin or leptin (116-130) treatment, and in the presence of bpV and either leptin, or leptin (116-130). Inhibition of PTEN mimicked and occluded the effects of leptin (116-130).

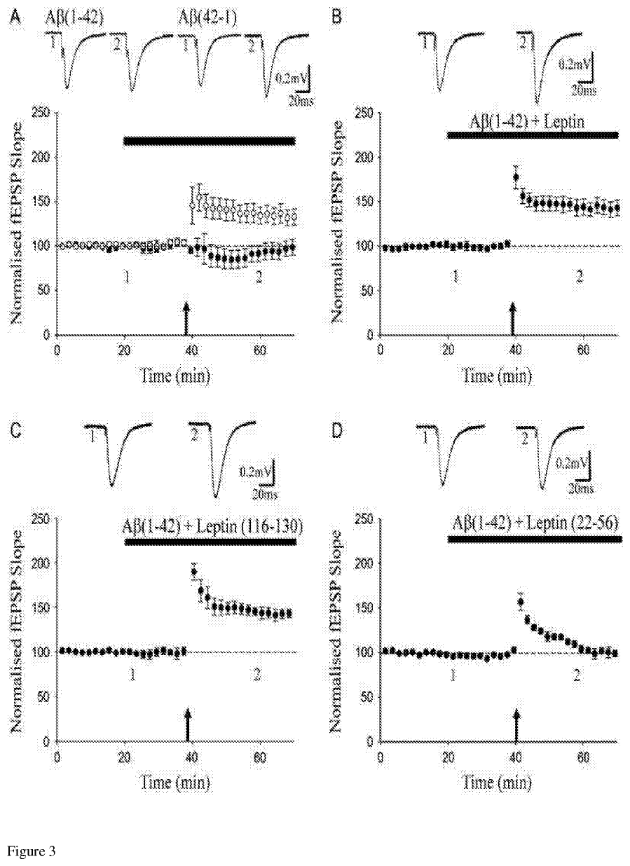

FIG. 3A, FIG. 3B, FIG. 3C and FIG. 3D show Leptin (116-130) inhibits the aberrant effects of amyloid-β Aβ₁₋₄₂ on hippocampal synaptic plasticity in juvenile slices. FIG. 3A: pooled data showing that HFS (indicated by the arrow) induces synaptic plasticity in Aβ₄₂₋₁-treated (open circles) slices, whereas Aβ₁₋₄₂ inhibits synaptic plasticity (filled circles). FIG. 3B: exposure to leptin prevented Aβ₁₋₄₂-inhibition of synaptic plasticity. Treatment with leptin (116-130; FIG. 3C) but not leptin (22-56; FIG. 3D) reversed Aβ₁₋₄₂-inhibition of synaptic plasticity.

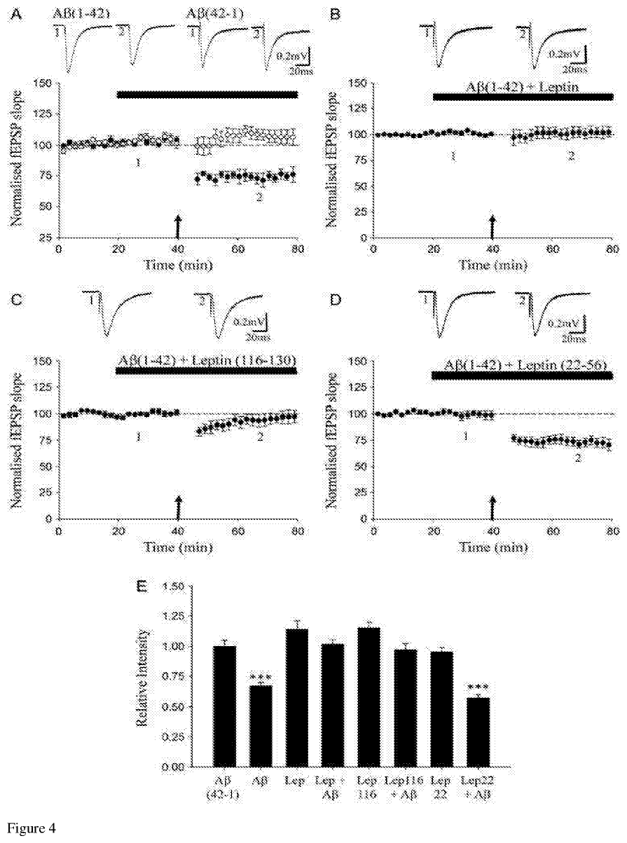

FIG. 4A, FIG. 4B, FIG. 4C, FIG. 4D and FIG. 4E show Leptin (116-130) prevents Aβ-induced AMPA receptor internalization and facilitation of hippocampal LTD. FIG. 4A: pooled data showing that the subthreshold low frequency stimulation (indicated by the arrow) failed to induce long-term depression (LTD) in Aβ42-1-treated (open circles) slices, whereas robust LTD (filled circles) is induced in Aβ1-42-treated juvenile slices. FIG. 4B: exposure to leptin prevented Aβ1-42-induced LTD. Similarly treatment with leptin (116-130; FIG. 4C) but not leptin (22-56; FIG. 4D) prevented Aβ1-42-induced LTD. FIG. 4E: pooled data showing relative changes in GluA1 surface labelling in cultured hippocampal neurons in control (Aβ42-1) conditions and after treatment with leptin, Aβ1-42, leptin (116-130), leptin (22-56) and in the presence of Aβ1-42 plus either leptin, leptin (116-130) or leptin (22-56), respectively. Leptin and leptin (116-130) prevent Aβ-driven internalization of GluA1.

FIG. 5A, FIG. 5B, FIG. 5C and FIG. 5D show that Leptin (116-130) prevents copper and A□-induced cell death in human SH-SY5Y cells. FIG. 5A: pooled data revealing that leptin and leptin (116-130) prevent LDH release induced by administration of 5 μM CuCl2. FIG. 5B: similar pooled data was obtained for cultures treated with 10 μM Aβ1-42. FIG. 5C: pooled data showing that in 5 μM CuCl2 treated cultures enhanced numbers of cells are detected with a crystal violet assay when cultures are co-treated with either leptin or leptin (116-130) with a similar trend observed when cultures were induced to die with 10 μM Aβ1-42 (FIG. 5D);

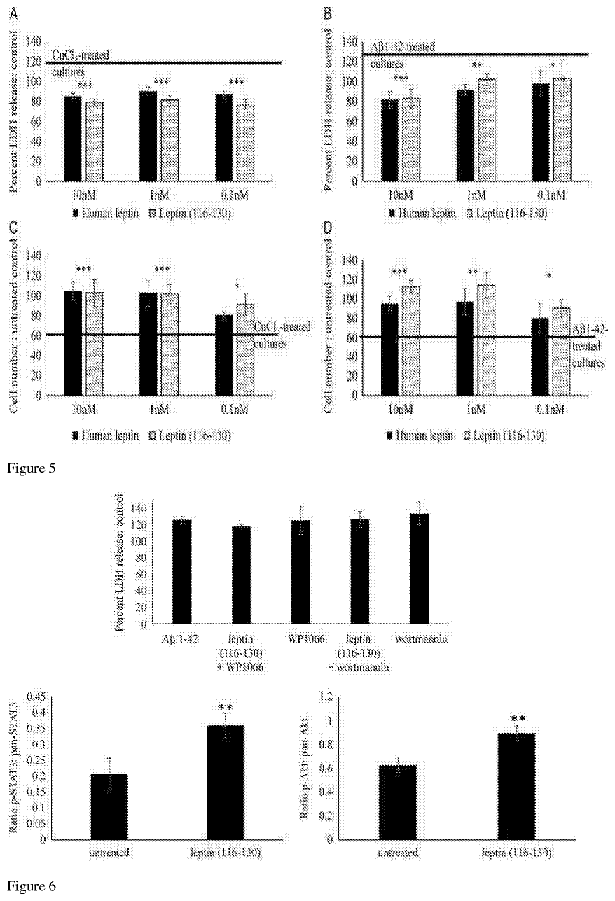

FIG. 6A, FIG. 6B and FIG. 6C show that the neuroprotective effects of leptin (116-130) involve activation of STAT3 and PI3-kinase-dependent signalling pathways. FIG. 6A: pooled data revealing that treatment of SH-SY5Y cells with the STAT-3 inhibitor WP1066 (5 μM) prevented leptin (116-130)-mediated neuroprotection from the effects of 10 μM Aβ1-42. FIG. 6A: pooled data from LDH assays demonstrating that the PI3-kinase inhibitor wortmannin (50 nM) attenuated leptin (116-130)-mediated neuroprotection against 10 μM Aβ1-42. Pooled data from ELISA assays showing that leptin (116-130) stimulates phosphorylation of STAT3 (FIG. 6B). Pooled data from ELISA assays showing that leptin (116-130) stimulates phosphorylation of Akt (FIG. 6C);

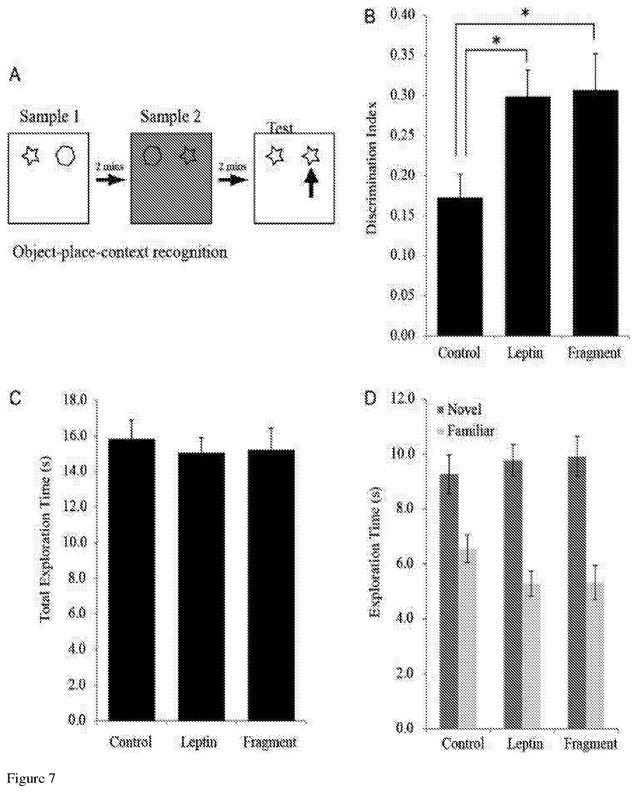

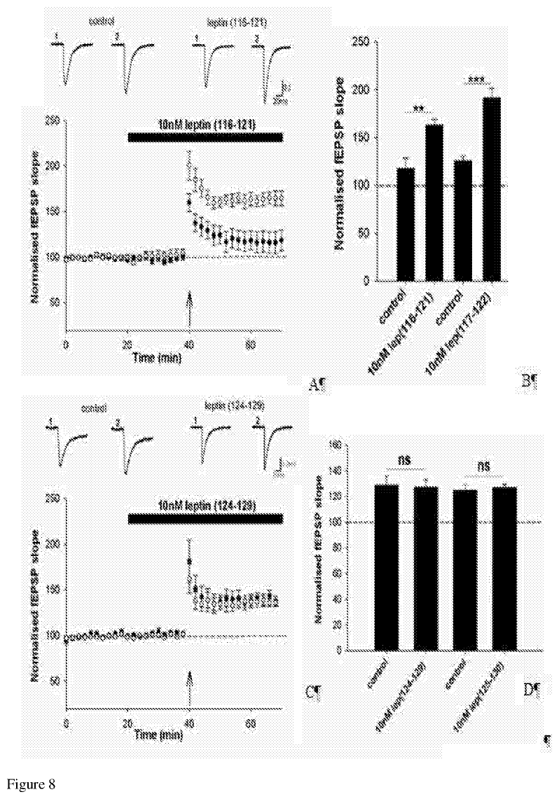

FIG. 7A, FIG. 7B, FIG. 7C and FIG. 7D show Leptin (116-130) enhances episodic-like memory. FIG. 7A: object-place-context task used to assess episodic-like memory. There are 2 sample phases in which mice are exposed to different copies of 2 different objects (star and hexagon) and allowed to explore for 3 min. In the test phase they see 2 new copies of 1 of the objects. The arrow points to the object that has not been previously seen in that place within that context. FIG. 7B: mean±SEM discrimination index for the 3 groups. *P<0.05. FIG. 7C: total exploration time in the test phase is not different between groups. FIG. 7D: exploration of the novel and familiar objects in the test phase; treatment with leptin or the leptin fragment (116-130) enhanced performance on the episodic-like memory test.

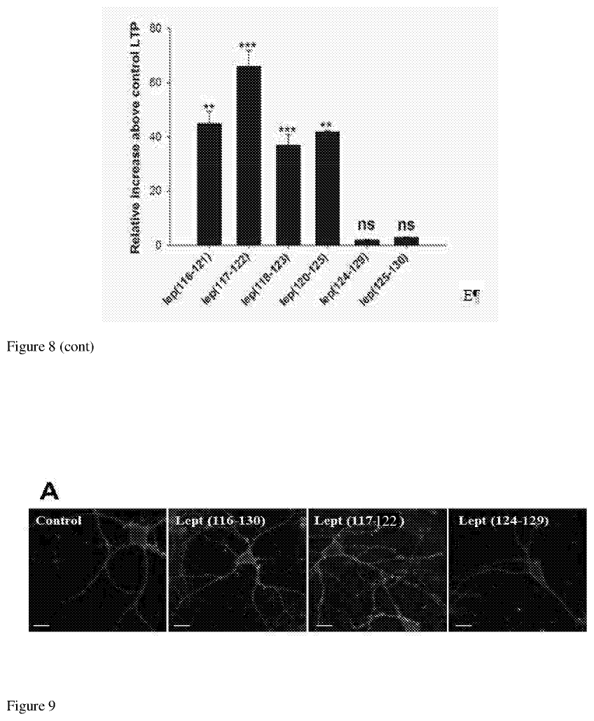

FIG. 8 shows Leptin (116-121) and (117-122) facilitate hippocampal LTP; Top panel: Application of leptin (116-121; open circle) significantly increased the magnitude of LTP compared to control (filled circle; n=6). Middle panel, In contrast, application of leptin (124-129; open circle) failed to alter the magnitude of LTP compared to control (filled circle; n=6). 2B, Lower panel; Histograms of pooled data showing the effects of the different hexamers on the magnitude of LTP in juvenile hippocampal slices. Leptin (116-121) and leptin (117-122) both increased the magnitude of LTP (2B), whereas leptin (124-129) and leptin (125-130) were without effect (2D). *** P<0.001; **P<0.01; *P<0.05.

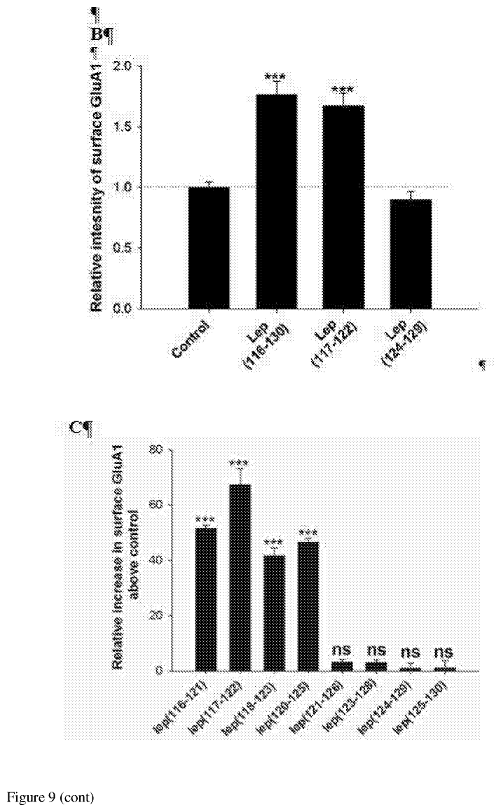

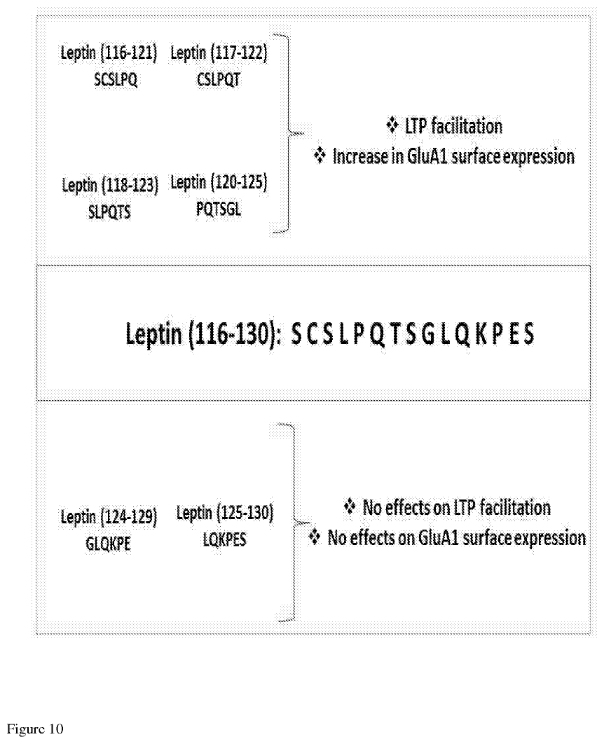

FIG. 9A and FIG. 9B show that Leptin (116-130) and leptin (117-122), but not leptin (124-129) enhance GluA1 surface expression in hippocampal neurons. FIG. 9A: representative confocal images of surface GluA1 expression in hippocampal neurons (8 DIV) in control conditions and following treatment with leptin (116-130), leptin (117-122) or leptin (124-129). GluA1 surface expression was detected using an antibody against a cell-surface epitope of GluA1 as previously (Moult et al, 2010). The scale bars represent 10 μm. FIG. 9B: pooled data (n=48) showing relative changes in control conditions and after exposure to leptin (116-130), leptin (117122) or leptin (124-129). Both leptin (116-130) and leptin (117-122) increased GluA1 surface labelling, whereas leptin (124-129) had no effect. *** P<0.001; **P<0.01; *P<0.05. C, Summary plot of the effects of the leptin fragments on AMPA receptor trafficking.

FIG. 10 is a summary of leptin fragment hexamers and their effects on hippocampal synaptic function.

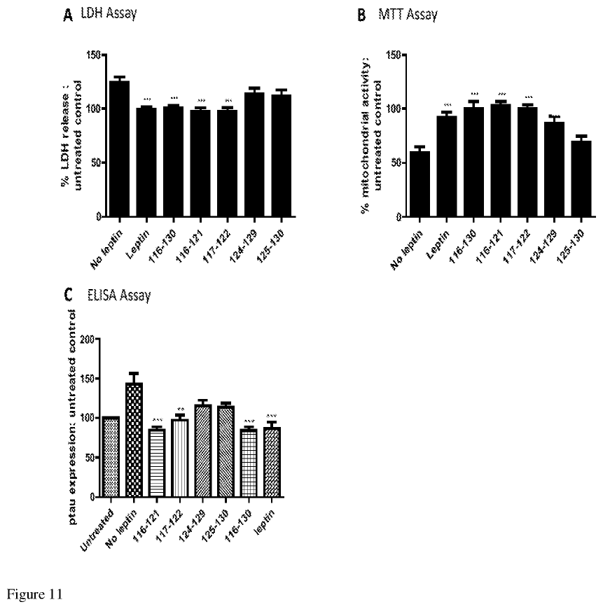

FIG. 11A, FIG. 11B, and FIG. 11C show Leptin 116-121 and 117-122 protect against Aβ₁₋₄₂-mediated cell death in differentiated SH-SY5Y human neuronal cells in vitro. FIG. 11A: histogram of pooled data obtained using an LDH assay which detects release of lactate dehydrogenase and therefore neuroprotection is indicated by a decrease in levels. Treatment with leptin and the leptin fragment (116-130) protects against Aβ-induced cell death induced by Aβ₁₋₄₂. This neuroprotective action is mirrored by the hexamers leptin (116-121) and leptin (117-122), but not leptin (124-129) and leptin (125-130). FIG. 11B: histogram of pooled data obtained using an MTT assay that monitors mitochondrial activity and therefore an enhanced level of activity reflects greater neuronal survival. Exposure to either leptin or leptin (116-130) protects against Aβ-induced cell death, and again this is mirrored by the hexamers leptin (116-121) and leptin (117-122), but not leptin (124-129) and leptin (125-130). FIG. 11C: data from and ELISA assay measuring phosphorylated tau expression in differentiated SH-SY5Y human neuronal cells treated with leptin, leptin (116-130) or the leptin hexamers. Treatment with leptin and the leptin fragment (116-130) protects against Aβ₁₋₄₂-mediated increase in p-tau, which is mirrored by the hexamers leptin (116-121) and leptin (117-122), but not leptin (124-129) and leptin (125-130). Data was analysed by one-way ANOVA with Tukey post hoc test; residuals were normally distributed. All significant differences are stated relative to Aβ₁₋₄₂ viability where: *** P<0.001; **P<0.01; *P<0.05; n=11 biological repeats (each biological data point representing the mean of 3 technical repeats).

Methods

Primary Neuronal Culture

Hippocampal cultures were prepared from neonatal Sprague Dawley rats as before (O'Malley et al. 2007). Briefly, neonatal Sprague Dawley rats (1-3 days old) were killed by cervical dislocation in accordance with Schedule 1 of the United Kingdom Government Animals (Scientific Procedures) Act, 1986 and the hippocampi removed. After washing in HEPES buffered saline (HBS) comprising (mM): NaCl 135; KCl 5; CaCl₂ 1; MgCl₂ 1; HEPES 10; D-glucose 25 at pH 7.4; cells were treated with protease Type X and Type XIV (0.5 mg/ml; Sigma) for 25 min at room temperature. Dissociated cells were plated onto sterile dishes (Falcon 3001) treated with poly-1-lysine (20 μg/ml; 1-2 h) and maintained in MEM with serum replacement-2 (Sigma) in a humidified atmosphere of 5% CO2 and 95% O2 at 37° C. for up to 3 weeks.

Human Neural Cell Line SH-SY5Y

The human neuroblastoma cell line, SH-SY5Y (ECACC, UK) was maintained in Dulbecco's Modified Eagle Medium supplemented with glucose (4500 μg/l) and 10% (v/v) cosmic calf serum (Fisher Scientific, UK) at 37° C. in a humidified atmosphere of 5% CO2, 95% air and allowed to reach 70-80% confluence before seeding. Cells (passage 10-18) were plated at 10 000 cells per well in 96 well tissue culture plates (Nunc, VWR, UK) and at a density of 2×10⁵ cells in 35 mm dishes for protein extraction. To induce differentiation, cells were cultured in DMEM supplemented with glucose (4500 μg/l), 1% (v/v) cosmic calf serum and 10 μM retinoic acid for 5 days. Thereafter they were incubated in DMEM supplemented with glucose (4500 μg/l), serum replacement 2 (2%; Sigma, UK) and 18 μM 5-fluorodeoxyuridine to inhibit proliferation of undifferentiated cells. The 50% of the medium was changed every 2-3 days and pharmacological treatment was carried out 7 days after switching to this medium. Reagents used were (from Sigma UK unless stated) 0.1-10 nM human leptin and leptin (116-130); 0.1-10 nM leptin (22-56; Bachem; Switzerland); 10 nM leptin (116-121) leptin (117-122), leptin (124-129) or leptin (125-130; all Severn Biotech, UK); 5 μM copper chloride; 10 mM Aβ₁₋₄₂, 5 μM WP1066 or 50 nM wortmannin. All treatments were added to the culture at the same time and survival assays were carried out after 96 h treatment. Protein samples for signaling ELISA were extracted 3 h after exposure to the relevant reagents and for biomarker expression after 72 hours.

Cell Survival Assays

The concentration of lactate dehydrogenase (LDH) in the culture medium or the mitochondrial activity within cells was used to monitor the level of cell death, as previously (Oldreive and Doherty 2010). In certain experiments, a Crystal violet assay was used to assess total cell number. Cells were fixed in neutral buffered formalin and washed 3 times in PBS prior to staining with 0.01% crystal violet acetate for 5 min. Plates were washed 5-10 times in dH2O and cells solubilised in 100 μl dimethyl sulfoxide (DMSO) before reading the absorbance on a Biohit BP100 plate reader. For all assays, data is expressed as percentage relative to control, untreated wells to normalize for differences in plating density between individual experiments.

ELISA for Cell Signalling Pathways and Phosphorylated Tau

Protein from cultures was extracted into 500 μl Tris-buffered saline containing protease inhibitor cocktail, and a Bradford assay used to determine protein concentration. Samples were diluted to give equal loading onto ELISA plates. Commercially available ELISA kits were used in accordance with the manufacturer's instructions to determine the ratio of pan-STAT3 to phospho-STAT3 (Sigma, UK) and pan-Akt to phospho-Akt (Sigma, UK) and the levels of alpha-tubulin and phosphorylated tau (Sigma, UK). Each protein sample was run in duplicate using samples derived from at least 5 biological repeats.

Surface Labelling of AMPA Receptors

To monitor GluA1 surface expression, immunocytochemistry was performed on hippocampal cultures (7-14 DIV) as before (Moult et al. 2010). Neurons were treated with agents for 20 min at room temperature (20-22° C.) before incubation with an antibody against an N-terminal region of GluA1 (sheep anti-GluR1; in house antibody against synthetic peptide (RTSDSRDHTRVDWKR) corresponding to 253-267 residues of GluR1; 1:100; Moult et al. 2010) at 4° C. Neurons were then fixed with 4% paraformaldehyde (5 min) before adding an appropriate fluorescently labelled secondary.

Electrophysiology.

Parasagittal hippocampal slices (300 μm) were prepared from either P13-21 or 12-24-week-old male Sprague Dawley rats as previously (Moult et al. 2010). Brains were rapidly removed and placed in ice-cold artificial CSF (aCSF; bubbled with 95% O₂ and 5% CO₂) containing the following (in mm): 124 NaCl, 3 KCl, 26 NaHCO₃, 1.25 NaH₂PO₄, 2 CaCl₂, 1 MgSO₄, and 10 d-glucose. Once prepared, parasagittal slices were allowed to recover at room temperature in oxygenated aCSF for 1 h before use. Slices were transferred to a submerged chamber maintained at room temperature and perfused with artificial cerebrospinal fluid at 2 ml min⁻¹. Standard extracellular recordings were used to monitor evoked field excitatory postsynaptic potentials (fEPSP) from stratum radiatum. The Schaffer collateral-commissural pathway was stimulated (constant voltage; 0.1 ms) at 0.033 Hz, using a stimulus intensity that evoked a peak amplitude ˜50% of the maximum. Synaptic potentials were low pass filtered at 2 kHz and digitally sampled at 10 kHz. The fEPSP slope was measured and expressed relative to baseline. Synaptic records are the average of 4 consecutive responses and stimulus artefacts are blanked for clarity. Recordings were made using an Axopatch 200B amplifier and analyzed using LTP v2.4 software. In synaptic plasticity studies, the degree of potentiation was calculated 30-35 min after HFS and expressed as a percentage of baseline±standard error of mean (SEM).

Hippocampal Neuron Culture

Hippocampal cultures were prepared as previously (Moult et al, 2010). Neonatal Sprague-Dawley rats (1-3 d old) were neonatal Sprague-Dawley rats were killed by cervical dislocation in accordance with the UK Animals (Scientific Procedures Act) 1986 legislation. Hippocampi were removed, and after washing in HEPES-buffered saline comprising (mM) 135 NaCl; 5 KCl; 1 CaCl2; 1 MgCl2; 10 HEPES; and 25 D-glucose at pH 7.40-4, were treated with papain (1.5 mg ml21; Sigma-Aldrich) for 20 min at 37° C. Dissociated cells were plated onto sterile dishes (35 mm diameter; Greiner Bio-One, Kremsmun-ster, Austria) treated with poly-D-lysine (20 μg ml⁻¹; 1-2 h). Cultures were maintained in serum replacement medium (SR2; Sigma-Aldrich) in a humidified atmosphere of 5% CO2 and 95% O2 at 37° C. for up to 2 wk.

Immunocytochemistry.

Immunocytochemistry was conducted on primary hippocampal cultures (7-14 DIV). Prior to labelling with antibodies, neurons were washed with HBS containing glycine (0.01 mM) and incubated with agents for 1 h at room temperature. Cultures were fixed with 4% paraformaldehyde for 5 min, then permeabilized with 0.1% Triton X-100 for 5 min. To label endogenous tau, neurons were incubated with a rabbit anti-tau (Ab-217; Genscript) primary antibody (1:200 dilution) at room temperature for 30 min, followed by incubation with an Alexa Fluor555 donkey anti-rabbit (1:250, Invitrogen) secondary antibody for an additional 30 min duration. To assess the % co-localisation of tau at synapses, neurons were dual labelled with an antibody against the synaptic marker, PSD-95 (mouse anti-PSD-95; 1:500, Thermo Fisher) followed by application of an Alexa Fluor488 goat anti-mouse secondary antibody (1:500, Invitrogen).

Phosphorylated tau was labelled with primary antibody rabbit anti-tau (Ab-396) polyclonal antibody (1:500, Gen Script) corresponding to phosphorylation site of serine 396 (Y-K-S^{P}-P-V). To visualise p-tau labelling, neurons were then incubated for 30 min with Alexa fluor555 donkey anti-rabbit IgG secondary antibody (1:250, Thermo Fisher Scientific). For synaptic localisation of p-tau, a secondary primary antibody was then added to compare p-tau localization relative to the synaptic marker, PSD95 (mouse anti-PSD-95, 1:500, Thermo Fisher Scientific) for 30 min. PSD-95 labelling was visualized by incubating with a goat anti-mouse Alexa Fluor 468-conjugated antibody (1:500; Thermo Fisher Scientific) for 30 min.

Imaging and Analyses

A Zeiss LSM510 confocal microscope was used for image acquisition and analysis. Images were obtained using a 10-s scan speed in single tracking mode or multi-tracking mode for dual labelling experiments. The intensity of immunostaining was measured off-line using Lasersharp software (Zeiss Lasersharp), whereby analysis lines of 50 μm length were drawn along randomly selected dendritic regions. All data were obtained from at least three different cultures from different animals. Imaging conditions including illumination intensity and photomultiplier gains were kept constant between treatments for each experiment. In addition, data were normalised relative to the mean fluorescence intensity obtained from control neurons. For synaptic co-localisation experiments, tau and PSD-95 positive immunostaining were compared. The number of tau-positive sites that co-localized with PSD-95 positive sites were counted and expressed as a percentage of total positive sites (peaks of intensity).

All data are presented as mean±SEM. Statistical analyses were performed using a Student independent t test for comparison of means (for % co-localization) and a one-way ANOVA with Tukey's post hoc test for comparisons between multiple groups. A value of p<0.05 was considered significant.

Cell Culture

The human neuroblastoma cell line, SH-SY5Y (ECACC, UK) was maintained in Dulbecco's Modified Eagle Medium supplemented with glucose (4500 μg/l) and 10% (v/v) cosmic calf serum (Fisher Scientific, UK) at 37° C. in a humidified atmosphere of 5% CO2, 95% air and allowed to reach 70-80% confluence before seeding. Cells (passage 10-18) were plated at 2×10⁵ cells on 13 mm borosilicate class coverslips or at 10 000 cells per well in 96 well tissue culture plates (Nunc, VWR, UK) prior to treatment. Reagents used were (from Sigma UK unless stated) 1 nM human leptin, leptin (116-121), leptin (117-122) and leptin (117-125); 0.0001-1 nm murine leptin fragments leptin (116-121), leptin (117-122) and leptin (116-130); 100 μM 6-hydroxydopamine; 10 μM CuCl₂; 1 mM Aβ₁₋₄₂.

Thioflavin S Staining

After 96 hours, cultures were fixed for 15 minutes in neutral buffered formalin, washed in phosphate buffered saline and stained with 0.05% Thioflavin S for 10 minutes prior to washing in phosphate buffered saline and mounting for visualisation on a fluorescent microscope. Relative fluorescent intensity per cell was determined using post hoc image analysis with Fiji software. Quantitative data is presented as the mean fluorescence per cell relative to control untreated cells.

Determination of Cell Viability

The concentration of lactate dehydrogenase (LDH) in the culture medium or the mitochondrial activity within cells was used to monitor the level of cell death, as previously (Oldreive and Doherty 2010). A Crystal violet assay was used to assess total cell number. Cells were fixed in neutral buffered formalin and washed 3 times in PBS prior to staining with 0.01% crystal violet acetate for 5 min. Plates were washed 5-10 times in dH2O and cells solubilised in 100 μl dimethyl sulfoxide (DMSO) before reading the absorbance on a Biohit BP100 plate reader. For all assays, data is expressed as percentage relative to control, untreated wells to normalize for differences in plating density between individual experiments.

Mitored Analysis of Mitochondrial Morphology

Cells were treated with pre-warmed cell culture medium containing 1 μM MitoRed ((9-[2-(4′-Methylcoumarin-7′-oxycarbonyl)phenyl]-3,6-bis(diethylamino)xanthylium chloride) and returned to the incubator for 45 minutes. Following fixation in NBF for 15 minutes, cells were washed 3 times in PBS. Coverslips were mounted in fluorescence mountant onto microscope slides and imaged on the Zeiss Axio MR2 microscope. The mean mitochondrial area and index of fragmentation (number of mitochondria: total area of mitochondrial material) were calculated using Fiji.

Results

Leptin (116-130) Facilitates NMDA Receptor-Dependent Hippocampal Synaptic Plasticity

It is known that NMDA receptors contribute little to basal excitatory synaptic transmission but synaptic activation of NMDA receptors is crucial for LTP induction at hippocampal synapses (Bliss and Collingridge 1993). Previous studies indicate that lep-tin enhances the magnitude of activity-dependent LTP in acute hippocampal slices (Oomura et al. 2006) and following direct administration into the hippocampus in vivo (Wayner et al. 2004). We have also shown that leptin facilitates NMDA receptor-dependent synaptic plasticity as leptin promotes conversion of short term potentiation (STP) to hippocampal LTP (Shanley et al. 2001). In order to compare the effects of leptin and the leptin fragments on synaptic plasticity in juvenile hippocampal slices (P13-21), a primed burst stimulation paradigm (5 trains of 8 stimuli at 100 Hz; Rose and Dunwiddie 1986) was used to induce STP, which returned to baseline levels within 30-35 min (n=4; FIG. 1A). In accordance with previous studies (Shanley et al. 2001), application of leptin (50 nM) prior to the stimulation paradigm converted STP into a persistent increase in synaptic transmission (155±6.1% of baseline at 40 min; n=4; P<0.05; FIG. 1A). Similarly, treatment with 50 nM leptin (116-130) facilitated synaptic plasticity as synaptic transmission was enhanced to 143±6.4% of baseline in leptin (116-130)-treated slices (n=4; P<0.05; FIG. 1B). In contrast, in slices exposed to 50 nM leptin (22-56), only STP was observed as synaptic transmission returned to baseline levels within 40 min (n=6; FIG. 1C).

Leptin (116-130) Induces Synaptic Plasticity at Adult Hippocampal CA1 Synapses

We have shown that leptin regulation of excitatory synaptic transmission is age-dependent. Thus in contrast to its effects in juvenile tissue, leptin (25 nM) induces a novel form of LTP in adult hippocampus (Moult and Harvey 2011). In order to verify if the leptin fragments mirror leptin action in adult tissue, the effects of the leptin fragments were examined in hippocampal slices from adult (12-24 weeks) rats. In accordance with previous studies (Moult et al. 2010; Moult and Harvey 2011), application of leptin (25 nM; 20 min) to adult slices rapidly enhanced synaptic transmission (to 188±13% of baseline; n=4; P<0.01; data not shown) which was sustained for the duration of recordings. Synaptic transmission was also markedly increased (to 140±13% of baseline; n=5; P<0.05; FIG. 2A) after treatment with 25 nM leptin (116-130), whereas application of 25 nM leptin (22-56) had no significant effect on synaptic efficacy (99±2% of baseline; n=4; P>0.05; FIG. 2B). These data indicate that leptin (116-130) mirrors the ability of leptin to enhance synaptic efficacy in adult hippocampus.

Leptin (116-130), But Not Leptin (22-56) Enhances the Surface Expression of GluA1

Trafficking of AMPA receptors to and away from synapses is crucial for various forms of activity-dependent synaptic plasticity (Collingridge et al. 2004). Our studies indicate that leptin regulates AMPA receptor trafficking as leptin promotes trafficking of GluA1 to hippocampal synapses (Moult et al. 2010). Moreover the ability of leptin to induce LTP in adult hippocampus requires the delivery of GluA1 to synapses by leptin (Moult et al. 2010). Thus, to assess if the leptin fragments also influence AMPA receptor trafficking processes, the surface expression of GluA1 was assayed in cultured hippocampal neurons (Moult et al. 2010). In agreement with previous studies, application of leptin (50 nM; 15 min) increased surface GluA1 expression to 184±7%; (n=36; P<0.001; FIG. 2C,D) compared with control hippocampal neurons. Similarly GluA1 surface expression was elevated (to 160±6% of control; n=36; P<0.001) after exposure to 50 nM leptin (116-130; FIG. 2C,D), whereas GluA1 surface expression (98±4% of control; n=36; P>0.05) was not altered after treatment with 50 nM leptin (22-56). These data indicate that leptin (116-130), but not leptin (22-56) mirrors the plasticity enhancing effects of leptin by increasing GluA1 surface expression in hippocampal neurons.

As excitatory synaptic strength is governed by the density of AMPA receptors expressed at synapses, the effects on synaptic AMPA receptors was examined by comparing the colocalization between surface GluA1 and synaptophysin immunolabelling in hippocampal cultures (Moult et al. 2010). In agreement with previous studies (O'Malley et al. 2007; Moult et al. 2010), leptin (50 nM; 30 min) increased synaptophysin staining to 144±9% of control (n=48; P<0.001) and it also enhanced the degree of colocalization between surface GluA1 and synaptophysin immunostaining from 43±5.4% to 62±4.4% (n=36; P<0.05; FIG. 2E). Similarly, in hippocampal neurons treated with leptin (116-130), synaptophysin staining was increased to 122±9% (n=36; P<0.05) and a significant increase (to 64±4.9%; n=36; P<0.01) in GluA1-synaptophysin colocalization was observed (FIG. 2E). Conversely, exposure to leptin (22-56) had no significant effect on either synaptophysin staining (105±9%; n=36; P>0.05) or the degree of colocalization (49±5.4%; n=36; P>0.05; FIG. 2E). These data indicate that leptin (116-130), but not leptin (22-56) promotes the trafficking of GluA1 to synapses in hippocampal neurons.

Inhibition of the phosphatase, PTEN underlies leptin-driven trafficking of GluA1 to synapses (Moult et al. 2010). In order to determine if similar leptin dependent signalling cascades mediate the actions of leptin (116-130), the effects of pharmacological inhibition of PTEN with bisperoxovanadium (bpV; Schmid et al. 2004) were assessed in hippocampal cultures. Application of bpV (50 nM; 30 min) increased GluA1 surface expression to 148±8% of control (n=36; P<0.001; FIG. 2F). In accordance with previous studies (Moult et al. 2010), leptin resulted in a significant increase in surface GluA1 labelling (140±7.2%; n=36; P<0.001; FIG. 2F). Similarly, GluA1 surface expression was enhanced (to 167±10% of control; n=36; P<0.001) after exposure to leptin (116-130). Moreover, treatment with bpV occluded the effects of either leptin or leptin (116-130) such that GluA1 staining was 131±6.6% and 148±8.1% of control (n=36; P<0.001; for both; FIG. 2F) in the presence of bpV and either leptin or leptin (116-130), respectively. These data indicate that the ability of leptin (116-130) to regulate GluA1 trafficking involves inhibition of PTEN.

Leptin (116-130), But Not Leptin (22-56) Reverses Aβ1-42 Inhibition of Hippocampal Synaptic Plasticity

Several studies indicate that soluble Aβ oligomers impair activity-dependent synaptic plasticity, as exposure to Aβ inhibits hippocampal LTP (Shankar et al. 2008; Li et al. 2009) and enhances LTD (Shankar et al. 2008). Moreover, our recent studies indicate that leptin reverses the detrimental effects of Aβ1-42 on both LTP and LTD (Doherty et al. 2013). Thus we assessed if either of the fragments mirrored the protective actions of leptin on hippocampal synaptic plasticity. Initially we determined that application of leptin prevented the acute effects of _{Aβ1-42} on LTP. In control slices, synaptic plasticity was induced using high frequency stimulation (HFS; 100 Hz 10 trains of 8 stimuli) which increased synaptic transmission to 127±3.4% of baseline (n=8; P<0.01). Similarly in slices treated with the inactive peptide _{Aβ42-1} (1 tiM; 40 min), an enhancement of synaptic transmission (132±8.8% of base-line) was induced (n=5; P<0.05; FIG. 3A). However, in accordance with previous studies (Doherty et al. 2013), exposure to Aβ1-42 not only blocked hippocampal synaptic plasticity (96±7.5% of baseline; n=5; P>0.05; FIG. 3A) but this effect was reversed by leptin (25 nM; 135±6.3% of baseline; n=4; P<0.05; FIG. 3B. Similarly, treatment with 25 nM leptin (116-130) before _{Aβ1-42} resulted in a robust increase in synaptic transmission (to 136±5.5% of baseline; n=6; P<0.05; FIG. 3C). In contrast, HFS failed to increase synaptic efficacy (107±3.3% of baseline; n=5; P>0.05) in slices exposed to Aβ1-42 and leptin (22-56; FIG. 3D), although some STP was observed. Thus these data indicate that like leptin, leptin (116-130) prevents the detrimental effects of _{Aβ1-42} on hippocampal synaptic plasticity.

Leptin (116-130) Reverses Aβ1-42-Induced LTD

It is known that oligomeric Aβ promotes the induction of LTD (Shankar et al. 2008) and that exposure to a low concentration of leptin (10 nM) prevents facilitation of hippocampal LTD by Aβ1-42 (Doherty et al. 2013). Thus we assessed if either of the leptin fragments mirror leptin action. Initially we verified that leptin prevented Aβ1-42-induced LTD. In agreement with previous studies (Doherty et al. 2013), application of the subthreshold LFS paradigm failed to induce LTD in vehicle-treated slices (94±5.6% of baseline; n=5; P>0.05), whereas robust LTD (73±3.8% of baseline; n=5; P<0.001) was induced in Aβ1-42-treated slices (FIG. 4A). Furthermore, leptin (10 nM) reduced the magnitude of Aβ1-42-induced LTD such that LFS depressed synaptic transmission to 101±5.3% of baseline in leptin-treated slices (n=4; P>0.05; FIG. 4B). To establish if the leptin fragments mirrored leptin action the effects of leptin (116-130) or leptin (22-56) were also examined. Application of 10 nM leptin (116-130) or leptin (22-56) had no significant effect on basal synaptic transmission (n=6 for leptin [116-130] and n=5 for leptin [22-56]). Application of 10 nM leptin (22-56) failed to alter the magnitude of Aβ₁₋₄₂-induced LTD such that synaptic transmission was depressed to 73±4.0% of baseline (n=5; P<0.001; FIG. 4D). In contrast, a significant reduction in the magnitude of Aβ₁₋₄₂-induced LTD (94±5.2% of baseline; n=5; P>0.05; FIG. 4C) was observed in hippocampal slices treated with leptin (116-130; 10 nM; 45 min) indicating that leptin (116-130) also inhibits facilitation of LTD by Aβ₁₋₄₂. Thus these data indicate that leptin (116-130), but not leptin (22-56) reverses Aβ₁₋₄₂-induced facilitation of LTD.

Leptin (116-130) Prevents Aβ-Induced Internalization of GluA1

Previous studies indicate that Aβ promotes internalization of the AMPA receptor subunit, GluA1 (Hsieh et al. 2006; Liu et al. 2010); an effect that is prevented by leptin (Doherty et al. 2013). To determine if the leptin fragments mirror this effect, the cell surface density of GluA1 was probed in cultured hippocampal neurons (Moult et al. 2010). In accordance with previous studies (Doherty et al. 2013), treatment with Aβ₁₋₄₂ (500 nM; 20 min) significantly attenuated (to 70±2% of control) GluA1 surface expression compared with control (Aβ₄₂₋₁-treated) hippocampal neurons (n=48; P<0.001; FIG. 4E). Application of a low concentration of leptin (10 nM) induced a small increase in GluA1 surface expression (113±7% of control; n=36; P<0.01). However, in leptin-treated neurons, Aβ₁₋₄₂ failed to significantly alter GluA1 surface expression (101±4% of control; n=36; P>0.05). In contrast, treatment with a low concentration (10 nM) of leptin (22-56) had no significant effect on GluA1 trafficking per se (n=36; P>0.05). Moreover, leptin (22-56) failed to prevent the effects of Aβ on GluA1 trafficking as surface GluA1 was reduced to 51±2% of control (n=36; P<0.001) in the presence of Aβ and leptin (22-56; FIG. 4E). Conversely, after exposure to leptin (116-130), Aβ₁₋₄₂ failed to significantly reduce GluA1 surface expression (97±4% of control; n=36; P>0.05). These data indicate that leptin (116-130) mirrors leptin action by preventing Aβ₁₋₄₂-induced internalization of GluA1 in cultured hippocampal neurons. Leptin (116-130) Prevents Copper and Aβ-Induced Cell Death

We have demonstrated previously that leptin attenuates cortical neuronal death triggered by Aβ₁₋₄₂ or divalent copper ions (Doherty et al. 2013). To determine whether leptin (116-130) has neuroprotective actions, the effects of leptin (116-130) on the viability of differentiated human neural cells (SH-SY5Y) was examined after exposure to either 5 μM CuCl2 or 10 μM Aβ₁₋₄₂. Cells were treated with the toxin alone or with a range of concentrations (10-0.1 nM) of leptin or leptin (116-130). Determination of membrane leakage by LDH assay revealed a significant reduction in LDH release after treatment with either leptin or leptin (116-130; both 0.1-10 nM). Thus for CuCl₂-treated cells, 10 nM leptin reduced LDH release by 39.5±2.73% compared with CuCl₂ alone (n=5; P<0.001; FIG. 5A); an effect that was mirrored by 10 nM leptin (116-130; 45.6±2.92% [n=5; P<0.001; FIG. 5A]). In Aβ₁₋₄₂ treated cells, leptin also significantly reduced LDH release by 31.8±13.2% (0.1 nM) and 47.9±7.45% (10 nM), respectively (n=5; P<0.001; FIG. 5B). Similarly, application of 0.1 nM or 10 nM leptin (116-13) decreased LDH release by 26.7±17.3% or 46.6±9%, respectively (n=5; P<0.05; FIG. 5B). Thus like leptin, treatment with leptin (116-130) reduces neuronal death in response to AD-linked toxins in vitro.

In parallel studies a crystal violet assay was used to verify these findings by assessing cell number. In CuCl₂-treated cells, there was a concentration-dependent increase in the survival of cells treated with either leptin or leptin (116-130). Thus, treatment with leptin (0.1 nM) resulted in a 16.7±3.4% increase in cell number and this increased to 43.4±9.2% in the presence of 10 nM leptin (n=5; P<0.01). Similarly, exposure to 0.1 or 10 nM leptin (116-130) increased cell number by 27.8±10.6% and 39.9±13.5%, respectively (n=5; FIG. 5C). Treatment with leptin (116-130) also mirrored leptin action by increasing cell viability in Aβ₁₋₄₂-treated cultures as cell number increased by 19.2±15% and 44.3±7.5% after treatment with 0.1 nM or 10 nM leptin, respectively (n=5 for each; P<0.01). Exposure to leptin (116-130) also resulted in significant increases in cell number (0.1 nM: 29.4±9.4% increase; 10 nM: 51.8±6.3% increase; n=5 for both; P<0.001; FIG. 5D).

As leptin (22-56) has biological activity in other systems, the specificity of the leptin (116-130) fragment in promoting cell survival was examined by determining whether leptin (22-56) inhibited neuronal death induced by Aβ₁₋₄₂. In contrast to leptin (116-130), treatment with leptin (22-56) had no effect on the viability of cells exposed to Aβ₁₋₄₂ (41.2±5.8% survival following Aβ₁₋₄₂ treatment and 48.6±9.3% in Aβ₁₋₄₂ with 10 nM leptin (22-56) treated cells; n=5; P>0.5; data not shown). These data reveal a potent neuroprotective effect of the leptin fragment (116-130) that is comparable to the survival actions of leptin. Moreover, this anti-apoptotic response is specific to leptin (116-130) as leptin (22-56) failed to influence neuronal viability.

The Neuroprotective Effects of Leptin (116-130) Involve Activation of STAT3 and PI3-Kinase-Dependent Signalling Pathways

Our previous studies indicate a crucial role for STAT3 and PI3-kinase/Akt signalling in the neuroprotective actions of leptin (Doherty et al. 2013). To determine whether leptin (116-130) acts via similar signalling cascades we examined the effects of pharmacological inhibitors of STAT3 (WP1066) or PI3-kinase (wortmannin). In Aβ₁₋₄₂-treated SH-SY5Y cells, application of either inhibitor significantly reduced the ability of leptin (116-130) to alleviate neuronal death. When neurons were treated with the STAT3 inhibitor, an 18.3±3.2% increase in LDH release in leptin (116-130) and Aβ₁₋₄₂-treated cultures was observed, which is similar to the 26.7±4.4% increase observed with Aβ₁₋₄₂ alone (n=5; P>0.5; FIG. 6A). Thus STAT3 inhibition blocks the neuroprotective actions of leptin (116-130), suggesting a role for STAT3 in this process. Furthermore, following inhibition of PI3-kinase with wortmannin an 26.9±9.8% increase in LDH release was observed in leptin (116-130) and Aβ₁₋₄₂-treated cells which is not significantly different from cells treated with Aβ₁₋₄₂ alone (n=5; P>0.5; FIG. 6A. Thus these data also indicate a role for PI3-kinase in mediating the neuroprotective actions of leptin (116-130).

To verify that leptin (116-130) directly activates these signaling pathways, SH-SY5Y cells were exposed to 1 nM leptin (116-130; 3 h) or left untreated prior to protein extraction for ELISA. The ratio of phosphorylated STAT3 to pan STAT3 increased markedly following leptin (116-130) administration (n=3; P<0.01; FIG. 6B. Similarly an increase in the ratio of phosphorylated Akt to pan Akt was observed following exposure to leptin (116-130; n=3; P<0.01; FIG. 6C). These data indicate that leptin (116-130) reduces cell death by a mechanism involving activation of STAT3 and PI3-kinase. Furthermore, exposure to leptin (116-130) resulted in a significant increase in the active components of these signalling cascades.

Leptin (116-130) Enhances Episodic-Like Memory

The current data demonstrate that leptin (116-130) enhances hippocampal synaptic plasticity mechanisms and has neuroprotective effects. To further assess its therapeutic potential we next asked if this fragment has similar cognitive enhancing properties to the whole leptin molecule. Previous studies indicate that leptin enhances hippocampal-dependent memory (Oomura et al. 2006; Farr et al. 2006), whereas resistance to lep-tin results in impaired spatial memory (Li et al. 2002). We used the object-place-context (OPC) recognition task which models human episodic memory, the first cognitive process to be compromised in the early stages of AD (Swainson et al. 2001). Performance on this task has been shown to be impaired in murine models of AD (Davis et al. 2013) and is compromised in animals with lesions of hippocampus (Langston and Wood 2010) and lateral entorhinal cortex (Wilson et al. 2013). The task is based on the object recognition paradigm and models the integrated aspect of human episodic memory by exposing rodents to novel combinations of objects, the spatial locations in which they are experienced and the contextual features of the environment (FIG. 7A; Eacott and Norman 2004). A total of 42 C57/BL6 mice were habituated to a testing environment and then trained on object recognition, object-place recognition and object-context recognition. Following training mice were tested on 4 days of the episodic-like OPC task. On these days mice were assigned to 1 of 3 groups (control, leptin, or fragment) and on each day mice were given 100 μl IP injections of saline, 7.8 nM/ml leptin, or 7.8 nM/ml leptin (116-130) 30 min prior to testing. One-way ANOVA on the discrimination indices during the first minute of the test phase revealed a significant effect of group (FIG. 7B; F (2,41)=4.318, P=0.02). Post hoc comparisons (Tukey's HSD) revealed that both the leptin and leptin (116-130) treated mice showed enhanced performance on the task relative to the control group (P<0.05) and did not differ from each other. One sample t-tests confirmed that all groups performed significantly better than chance level performance (P<0.01). Finally analysis of the overall exploration time in both sample and test phases of the task revealed no change in total levels of exploration between groups demonstrating that neither leptin nor leptin (116-130) produced a non-specific change in exploration behaviour (FIG. 7C; P>0.05). The increased discrimination index was driven by an increase in exploration of the novel object combined with a decrease in the exploration of the familiar object (FIG. 7D). Together these data indicate that leptin (116-130) mirrors leptin's action by enhancing performance in episodic-like memory tasks.

Leptin (116-121) and Leptin (117-122) Facilitate Hippocampal LTP.

To determine if the leptin hexamers influence the magnitude of activity-dependent synaptic plasticity, standard extracellular recording techniques were used to assess the effects of leptin (116-121, 117-122, 118-123, 120-125, 124-129 and 125-130) on the magnitude of LTP induced by high frequency stimulation (100 Hz, 1 s) in acute hippocampal slices. In control slices, application of the HFS protocol resulted in robust LTP (117±10.8% of baseline; n=5; p<0.05; FIG. 8). Exposure of slices to 10 nM leptin (116-121) for 50 min prior to HFS also resulted in facilitation of LTP (to 162±6.3% of baseline; n=5; p<0.05; FIG. 8), and this was significantly greater than control LTP (p<0.05). Similarly, treatment with leptin (117-122) also facilitated LTP such that the magnitude of LTP was 191±19.7% of baseline (n=6; p<0.05) and this effect was significantly greater than control LTP (125±4.9% of baseline; n=5; p<0.05) evoked in interleaved slices. In parallel studies, application of leptin (118-123; 158±3.8%; n=7; p<0.05; FIG. 8) or leptin (120-125; to 163±8.8% of baseline; n=5; p<0.05) also facilitated LTP, compared to control LTP (FIG. 8). In contrast application of either 10 nM leptin (124-129) or leptin (125-130) failed to facilitate LTP as the magnitude of LTP was 137±5.6% of baseline (n=5; p>0.05; FIG. 8) and 137±2.4% (n=5; p>0.05) in the presence of leptin (124-129) and leptin (125-130), respectively, and this was not significantly different to LTP observed in control conditions. Thus of the fragments tested (FIG. 8), leptin (116-121), leptin (117-122), leptin (118-123) and leptin (120-125) all mirrored the actions of leptin and enhanced the magnitude of LTP, whereas leptin (124-129) and leptin (125-130) failed to alter the magnitude of LTP.

Leptin (116-121) and Leptin (117-122) Increase the Surface Expression of GluA1 in Hippocampal Neurons.

We have shown that leptin (116-130) increases the surface expression of GluA1 in hippocampal neurons (Malekizadeh et al, 2016), which mirrors the action of the whole leptin molecule. Thus to determine if the leptin hexamers are also capable of influencing AMPA receptor trafficking processes, the effects of leptin (116-121, 117-122, 118-123, 120-126, 122-128, 124-129 and 125-130) on the surface expression of GluA1 was assessed using immunocytochemical approaches in hippocampal neurons. Application of 10 nM leptin (116-121) for 15 min increased GluA1 surface expression to 146±9% of control (p<0.001; n=36; FIG. 9A). Similarly, exposure of neurons to 10 nM leptin (117-122) also increase surface GluA1 to 167±10% of control (p<0.001; n=36; FIG. 9A). Likewise treatment of hippocampal neurons with either leptin (118-123) or leptin (120-125) also increased the surface expression of GluA1 in hippocampal neurons (FIG. 9B and FIG. 9C). In contrast application of leptin (124-129) had no significant effect on GluA1 surface labelling (89±6% of control; p>0.05; n=48; see FIGS. 9B, 9C Likewise treatment with leptin (125-130) failed to significantly alter the surface expression of GluA1 (98±5% of control; n=36; p>0.05; FIG. 9C nor did treatment with either leptin (121-126) or leptin (122-128; FIG. 9C). Thus like leptin and leptin (116-130), treatment with leptin (116-121) or leptin (117-122) increase the surface expression of GluA1 suggesting potential cognitive enhancing action. Leptin (116-121) and leptin (117-122) mirror the neuroprotective actions of leptin and leptin (116-130)

To determine whether the leptin hexamers described above had neuroprotective actions, the effects of leptin (116-121, 117-122, 124-129 and 125-130) on the viability of differentiated human neural cells (SH-SY5Y) were examined after exposure to 10 μM Aβ1-42. Cells were treated with the toxin alone or 10 nM of leptin, leptin (116-130) or the listed hexamers. Determination of membrane leakage by LDH assay revealed a significant reduction in LDH release after treatment with either leptin or leptin (116-130; both 10 nM). Furthermore, leptin (116-121) and leptin (117-122) mirrored this neuroprotective effect but leptin (124-129) and leptin (125-130) did not. Thus for Aβ31-42-treated cells, 10 nM leptin and 10 nM leptin (116-130) significantly reduced LDH release by 25.3±2.5% and 24.0+2.2*% respectively compared with Aβ₁₋₄₂ alone (n=11; P<0.001; FIG. 11A); an effect that was mirrored by 10 nM leptin (116-121) and leptin (117-122); 27.1±3.1% and 27.4±3.5% (n=11; P<0.001; FIG. 11A), but not by 10 nM leptin (124-129) or leptin (125-130) (n=11; P>0.5; FIG. 11A). Thus like leptin and leptin (116-130), treatment with leptin (116-121) or leptin (117-122) reduces neuronal death in response to AD-linked toxins in vitro.

To confirm the findings of the LDH assay, cells were treated with Aβ₁₋₄₂ alone or with a 10 nM of leptin, leptin (116-130) or the listed hexamers, and mitochondrial activity, as a measure of cell viability, determined by MTT assay. A significant increase in mitochondrial activity was detected following treatment with either leptin or leptin (116-130; both 10 nM). Furthermore, leptin (116-121) and leptin (117-122) mirrored this neuroprotective effect but leptin (124-129) and leptin (125-130) did not. Thus for Aβ₁₋₄₂-treated cells, 10 nM leptin and 10 nM leptin (116-130) increased mitochondrial activity by 32.6±5.2% and 40.9+5.8% respectively compared with Aβ₁₋₄₂ alone (n=11; P<0.001; FIG. 11B); an effect that was mirrored by 10 nM leptin (116-121) and leptin (117-122); 43.61±3.9% and 40.5±3.7*% (n=11; P<0.001; FIG. 11B), but not by 10 nM leptin (124-129) or leptin (125-130) (n=11; P>0.5; FIG. 11B). This reinforces the findings from the LDH assay that similar to either leptin or leptin (116-130), treatment with leptin (116-121) or leptin (117-122) reduces neuronal death in response to AD-linked toxins in vitro.

Leptin (116-121) and Leptin (117-122) Mirror the Ability of Leptin and Leptin (116-130) to Reduce the Expression of the AD-Linked Biomarker Phosphorylated Tau (p-Tau)

Hyper-phosphorylation of tau is the underpinning mechanism of the development of neurofibrillary tangles—one of the key pathological feature of AD. Human SH-SY5Y neuronal cells were exposed to Aβ1-42 alone or in combination with 10 nM leptin, leptin (116-130), leptin (116-121), leptin (117-122), leptin (124-129) or leptin (125-130). Protein was extracted for ELISA assay and the ratio of p-tau to the house-keeping protein α-tubulin determined (FIG. 11C). Treatment with 10 nM leptin, leptin (116-130), leptin (116-121) or leptin (117-122) significantly decreased the Aβ₁₋₄₂-mediated increase in p-tau in these cells. In contrast leptin (124-129) and (125-130) did not. Thus for Aβ₁₋₄₂-treated cells, 10 nM leptin and 10 nM leptin (116-130) reduced p-tau expression by 56.2±8.0% and 58.9+4.6% respectively compared with Aβ1-42 alone (n=5; P<0.001; FIG. 11C); an effect that was mirrored by 10 nM leptin (116-121) and leptin (117-122); 58.1±4.0% and 45.48±6.1% (n=5; P<0.001 and P<0.01 respectively; FIG. 11C), but not by 10 nM leptin (124-129) or leptin (125-130)(n=5; P>0.5; FIG. 11C). These data reveal that leptin (116-121) and leptin (117-122) mirror the effects of leptin and leptin (116-130) in decreasing the expression of this AD-linked biomarker.

Previous studies have shown that exposure to amyloid beta (Aβ) promotes phosphorylation of tau, and that leptin protects against this aberrant action of Aβ in our models of AD [6]. Recent evidence indicates that treatment with oligomeric Aβ increases translocation of tau to synapses and this has been linked to synaptic dysfunction and ultimately cognitive impairments.

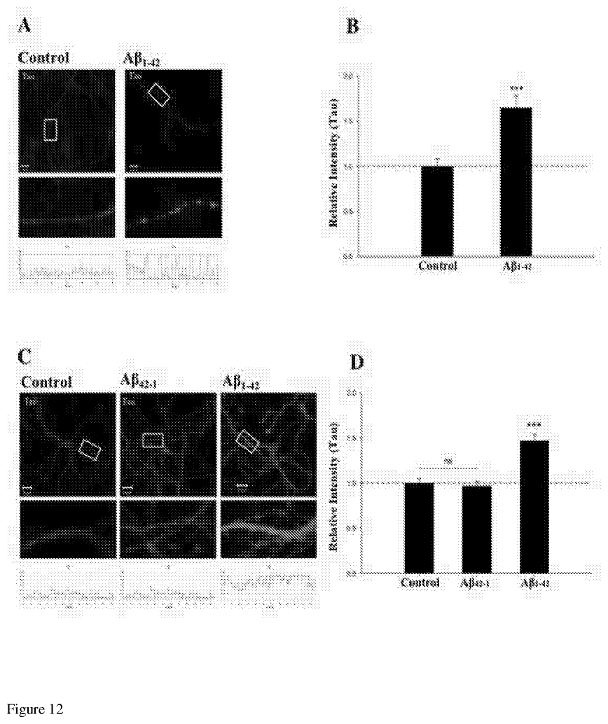

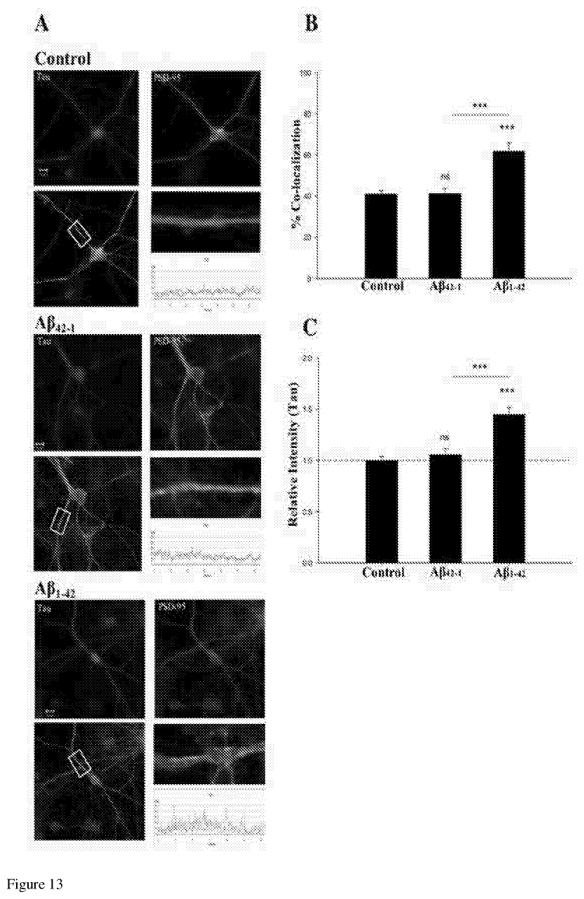

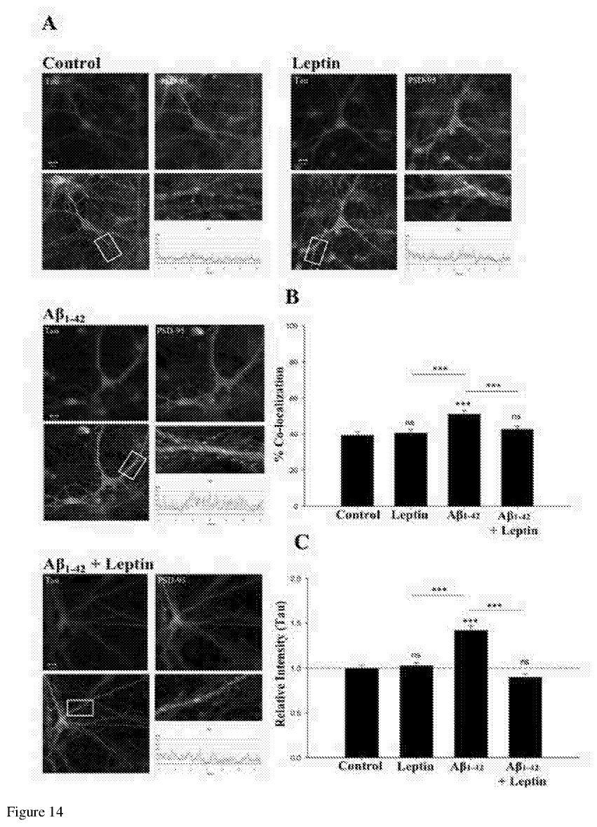

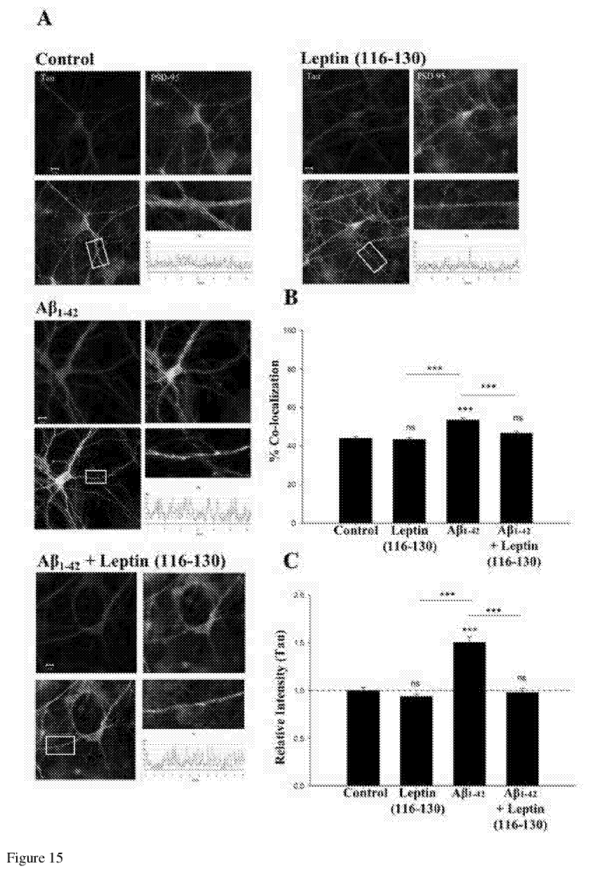

In accordance with existing data, we have set up and characterised a cellular model in hippocampal neurons that mirrors the aberrant trafficking of tau to synapses. In this model chronic treatment with Aβ results in increased expression of tau at dendrites and specifically trafficking of tau to synapses where it is likely to interfere with normal excitatory synaptic transmission (see FIGS. 12, 13). In this model of tau-related synaptic dysfunction, treatment with leptin protects against the Aβ-driven increase in the dendritic levels of tau, and it prevents tau trafficking to synapses (FIG. 14). Moreover, this protective effect of leptin is mirrored by the leptin fragment (116-130; FIG. 15).

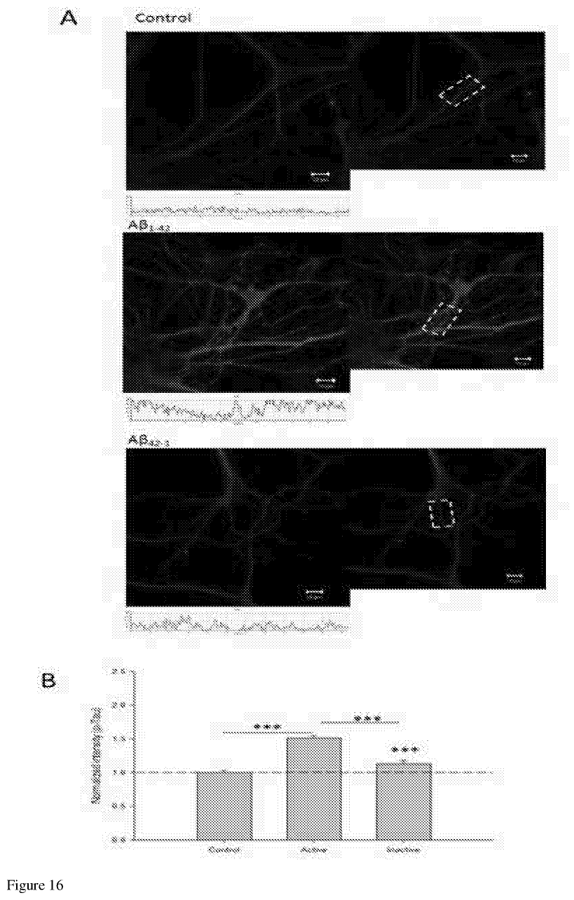

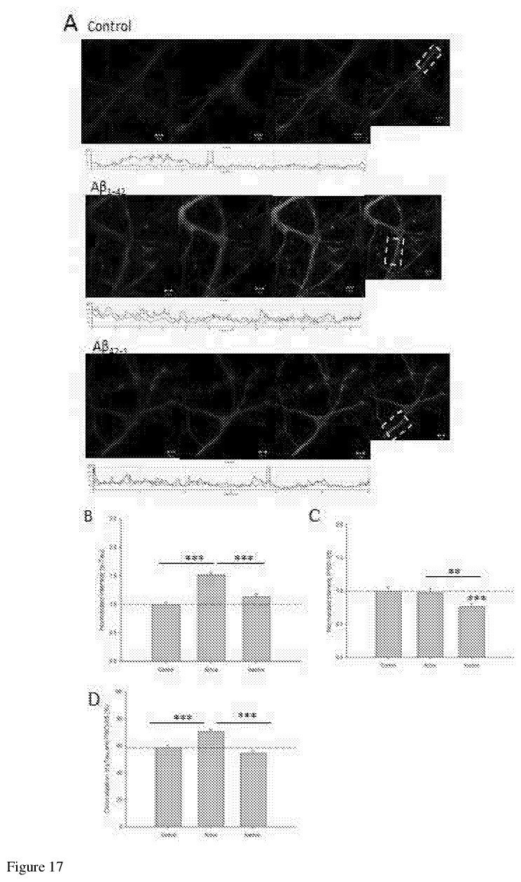

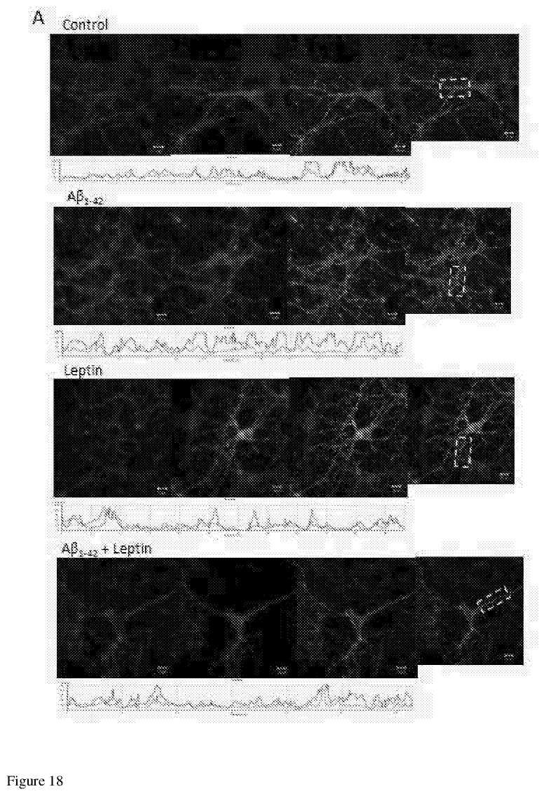

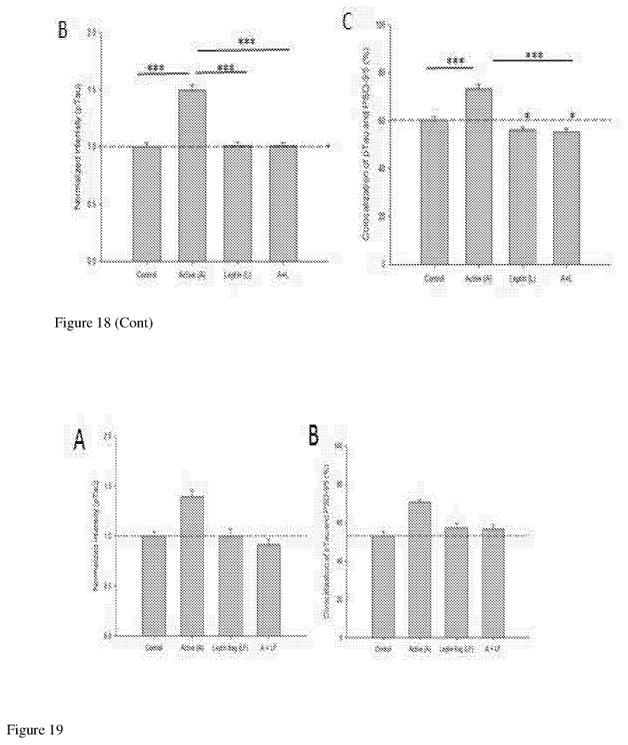

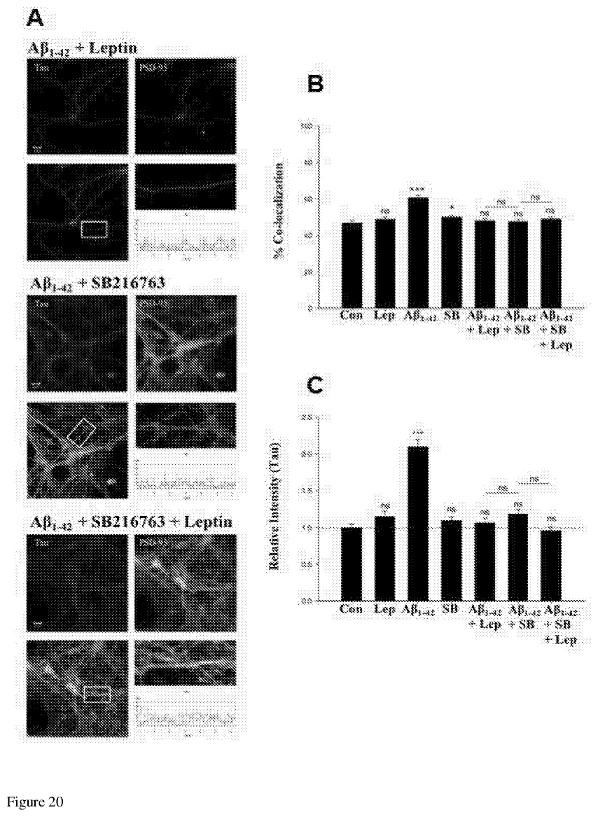

Previous studies have indicated that phosphorylation of tau is a key event that occurs in AD, and tau phosphorylation is also linked to its increased expression at synapses. In accordance with this, exposure to Aβ increases the dendritic levels of p-tau, and in particular the synaptic levels of p-tau are increased after exposure to Aβ (FIGS. 16, 17). Treatment with leptin and leptin (116-130) also protects against Aβ-driven increase in p-tau, as the dendritic levels of p-tau are reduced by leptin or leptin (116-130). Moreover, treatment with leptin or leptin (116-130) also prevents the ability of βP to traffic p-tau to synapses (FIG. 18, 19). As GSK-3β is known to be a key enzyme involved in tau phosphorylation, the role of this signalling pathway has also been explored. We find that inhibitors of GSK-3β prevent the neuroprotective actions of leptin and leptin (116-130; FIG. 20) suggesting that leptin driven inhibition of GSK-3β is likely to be the pathway involved in preventing tau phosphorylation and subsequent trafficking to hippocampal synapses.

Murine Leptin116-130

Previously we have shown neuroprotection in models of Alzheimer's Disease (AD)[1].

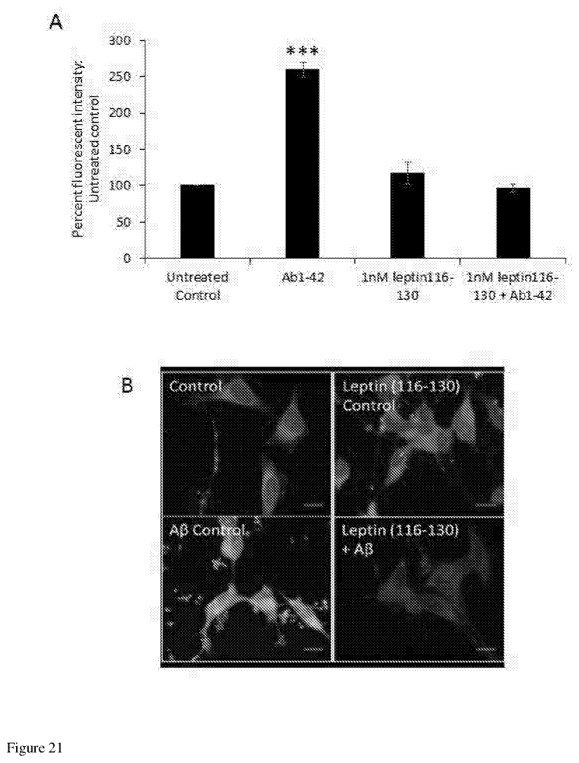

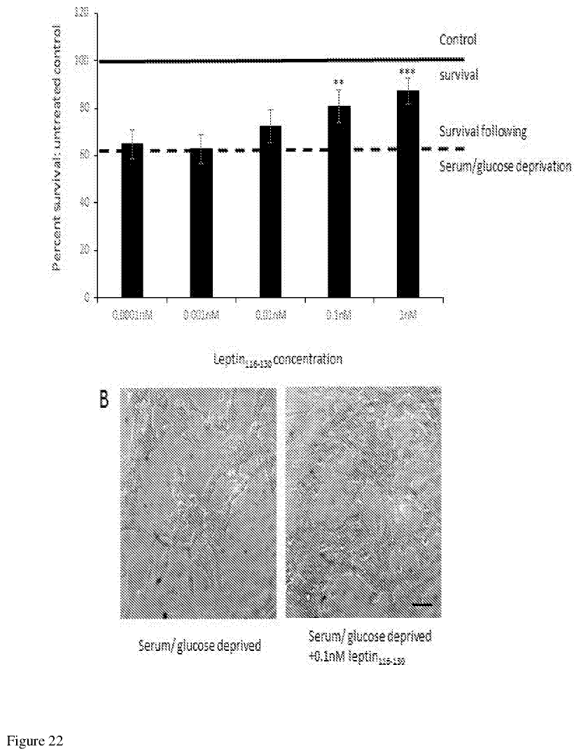

In accordance with the existing data we have demonstrated that leptin116-130 prevents the accumulation of amyloid beta following seeding of cultures with amyloid (FIG. 21). This fortifies our existing evidences that leptin116-130 has potent anti-AD effects in empirical models.

We have expanded upon our AD data to consider the potential of leptin116-130 to demonstrate neuroprotection in an in vitro model of ischemic stroke. Thus, an emerging model of stroke related neuronal death is the deprivation of serum and glucose from cultures of neural cells [New Ref]. Under these conditions we see significant neuroprotection by leptin116-130 (FIG. 22). This reveals a novel neuroprotective role of this fragment.

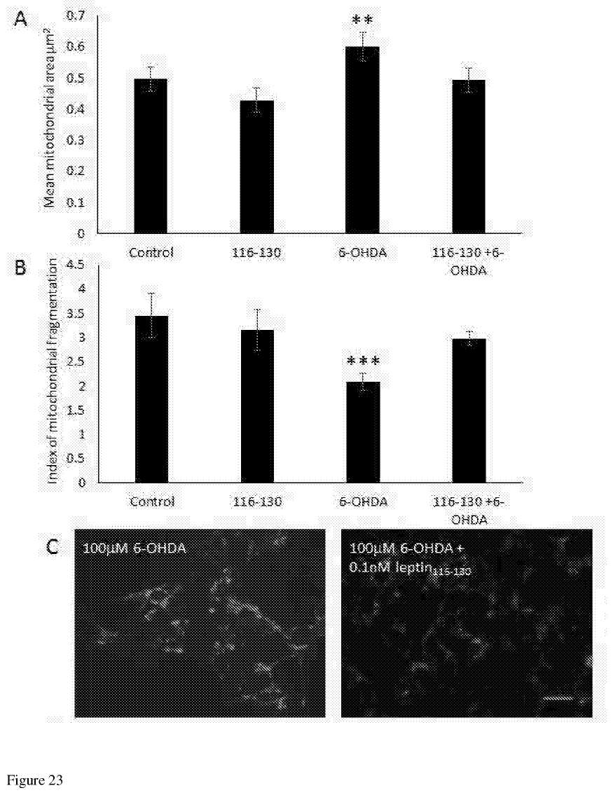

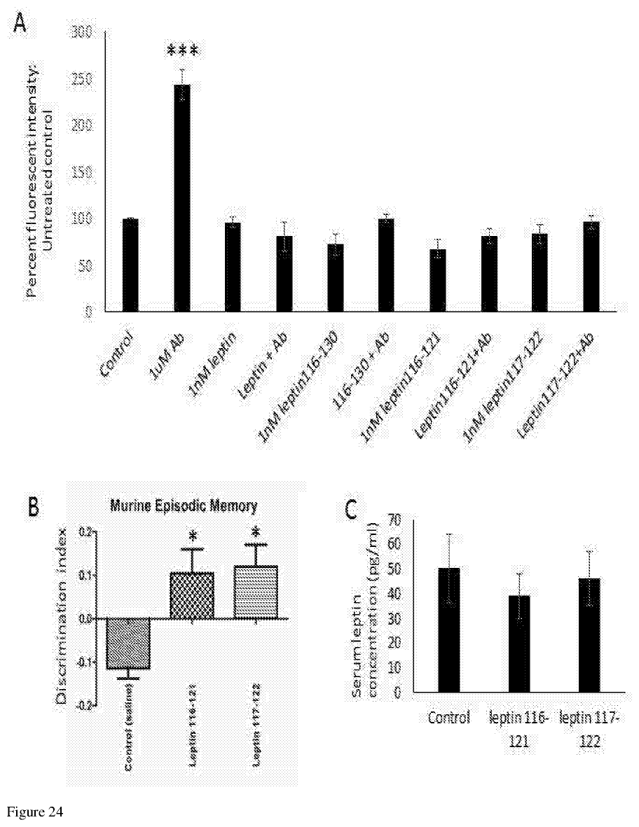

In a cellular Parkinson's Disease (PD) model, leptin116-130 prevents mitochondrial swelling and clumping in response to 6-hydroxydopamine (6-OHDA; FIG. 23), which is in keeping with earlier research on the full-length leptin molecule, which demonstrates robust neuroprotection from dopaminergic neurotoxins[7]. The current findings show the prevention of 6-OHDA-mediated neuronal swelling by leptin116-130 as demonstrated by the reduction in mean mitochondrial area when the fragment is present. In addition, 6-OHDA induces mitochondrial clumping as elucidated by the decrease in the fragmentation index of the mitochondrial pool in the presence of this neurotoxin. This is prevented by leptin116-130. Thus leptin116-130 prevents mitochondrial dysfunction in empirical models of PD.

Taken together these data strengthen the findings that leptin116-130 can protect against neuronal death and dysfunction in AD. Excitingly we have also expanded upon this existing knowledge to reveal the potential for a more general beneficial effect in other neurodegenerative conditions.

Murine Leptin Hexamers Based on Leptin₁₁₆₋₁₃₀

The data presented above reported pro-survival effects of leptin116-121 and leptin117-122 in AD models.

Building on these findings we have evaluated the potential for these hexamer fragments to ameliorate amyloid propagation after initial seeding with 1 μM Aβ1-42. We have demonstrated that leptin116-121 and leptin117-122 prevent the accumulation of amyloid beta following seeding of cultures with amyloid (FIG. 24A).

Further to this we have evaluated the effects of these hexamer peptides on episodic memory in mice. There is an 8 fold increase in episodic memory performance following treatment with leptin116-121 or leptin117-121 (FIG. 24B). This implies that both leptin hexamers enhance the ability of the mice to recognise the presence of a novel object within the test arena.

Blood samples taken 24 hours after injection showed no significant alterations in circulating leptin levels following any of the treatments revealing no long-term effects of the hexamers on endogenous leptin production (FIG. 24C).

Thus hexamer fragments of leptin₁₁₆₋₁₃₀ mirror the effects of leptin and of leptin₁₁₆₋₁₃₀ validating their further investigation as potential small peptide therapeutics.

Humanised Leptin Fragments Based on leptin₁₁₆₋₁₃₀

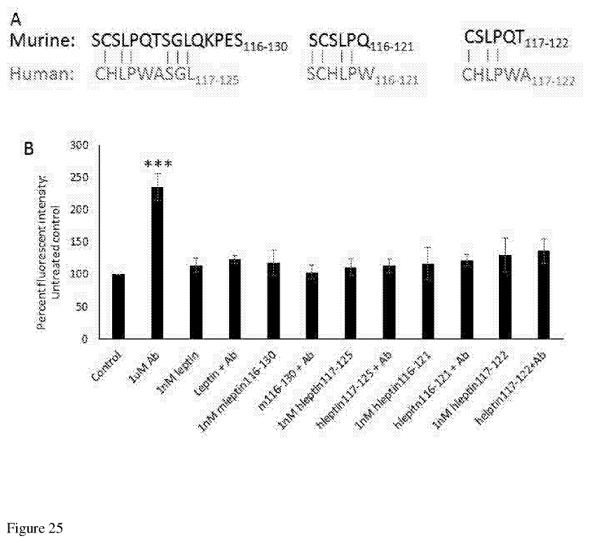

As all fragments tested above have been based on murine leptin116-130, we have designed and synthesised 3 human leptin fragments, hleptin117-125, hleptin116-121 and hleptin117-122 (FIG. 25A).

Using thioflavin S staining as before, we have demonstrated that hleptin117-125, hleptin116-121 and hleptin117-122 prevent the accumulation of amyloid beta following seeding of cultures with amyloid (FIG. 25B).

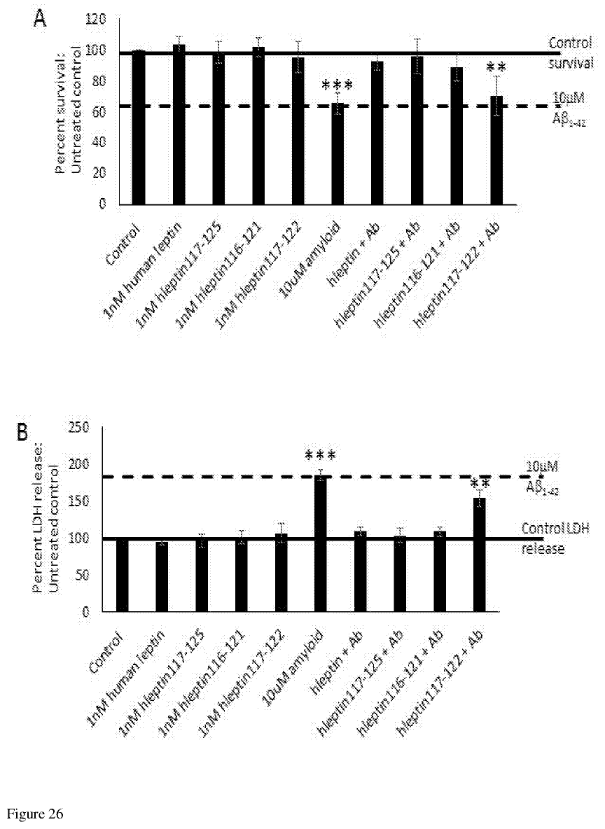

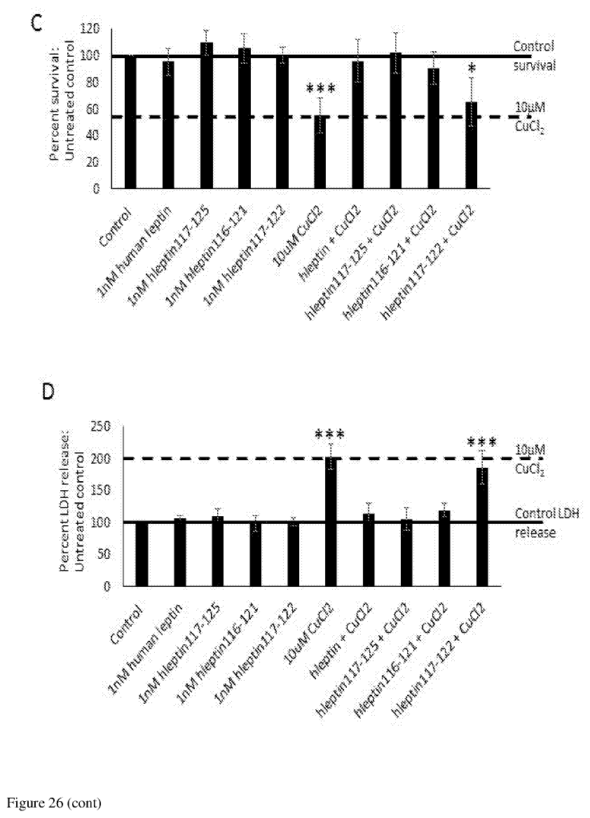

Furthermore both hleptin117-125 and hleptin116-121 prevent neuronal loss in vitro in response to either 10 μM amyloid betal-42 (FIGS. 26A and 26B) or 10 μM copper chloride (FIGS. 26C and 26D). This mirrors the action of murine leptin116-130.

These data reveal that humanised forms of target sequences within leptin116-130 demonstrate potent neurobeneficial effects in vitro. Crucially these sequences are amenable to peptide modification via halogenation, which the murine sequence is not (see below) and therefore open the possibility of peptide modification and stabilisation by this route.

Peptide Modification

Halogenation and cyclisation: Target sequences for halogenation should ideally contain a tryptophan and there is no such residue in murine leptin₁₁₆₋₁₃₀. Therefore, this work is focused on the human sequences, and to date 3 sequences (ₕleptin₁₁₇₋₁₂₅, ₕleptin₁₁₆₋₁₂₁, and ₕleptin₁₁₇₋₁₂₂) containing a 7-bromo-tryptophan have been synthesised by Severn Biotech, UK. Second generation peptides with alternative bromo-tryptophans and/or which have been cyclised are also being synthesised.

Discussion

It is well established that the hormone leptin circulates in the plasma and enters the brain via transport across the blood brain barrier. In the hypothalamus, leptin plays a major role in regulating food intake and body weight (Spiegelman and Flier 2001). However, the central actions of the hormone leptin are not restricted to the hypothalamus and the regulation of energy homeostasis. Indeed, a number of extrahypothalamic brain regions, including the hippocampus display high levels of leptin receptor expression (Irving and Harvey 2014). Leptin mRNA and protein are also highly expressed in the hippocampal formation (Morash et al. 1999) and emerging evidence suggests brain-specific production of leptin (Eikelis et al. 2006). Thus, it is likely that a combination of locally released leptin as well as peripherally derived leptin reach hippocampal synapses and can influence synaptic function. Indeed, numerous studies indicate that leptin has potential cognitive enhancing properties as it readily facilitates the cellular events underlying hippocampal learning and memory. Thus, leptin has rapid effects on activity-dependent synaptic plasticity, glutamate receptor trafficking and dendritic morphology (Irving and Harvey 2014). In addition, several studies have identified neuroprotective effects of leptin as the viability of central and peripheral neurons is markedly influenced by this hormone (Weng et al. 2007; Doherty et al. 2008; Guo et al. 2008). Recent clinical evidence has established a link between circulating leptin levels and the incidence of AD (Power et al. 2001; Lieb et al. 2009) that has fueled the possibility of using the leptin system as a novel therapeutic target in AD. Indeed, treatment of various AD models with leptin prevents the detrimental effects of Aβ that occur at both early and late stages of the disease (Fewlass et al. 2004; Farr et al. 2006; Doherty et al. 2013). However, as leptin is a very large peptide, developing small leptin-like molecules may be a better therapeutic approach. Several fragments of the leptin peptide are biologically active and mirror the anti-obesity effects of leptin (Grasso et al. 1997; Rozhayskaya-Arena et al. 2000; Grasso et al. 2001). However, the cognitive enhancing and neuroprotective effects of the leptin fragments are not known. Here we provide the first compelling evidence that leptin (116-130), but not leptin (22-56), has a potent effect on hippocampal synaptic function as it promotes trafficking of AMPA receptors to synapses and facilitates hippocampal synaptic plasticity. Moreover in cellular models that mimic amyloid toxicity, leptin(116-130), but not leptin (22-56) prevents the aberrant effects of Aβ on hippocampal synaptic function and neuronal viability. These findings indicate that one particular leptin fragment, namely (116-130), mirrors the beneficial actions of leptin in preventing the detrimental effects of Aβ at the early and late stages of AD. Finally we have shown that the leptin fragment that enhances hippocampal synaptic plasticity and has neuro-protective effects, namely leptin (116-130), is also a cognitive enhancer as it improves performance on tests of episodic memory.

Here we show that, in accordance with previous studies (Shanley et al. 2001; Wayner et al. 2004), NMDA receptor-dependent synaptic plasticity is enhanced by leptin as treatment with leptin promoted conversion of STP into a persistent increase in synaptic transmission in juvenile hippocampal slices. Similarly exposure to the leptin fragment (116-130) readily facilitated synaptic plasticity as an increase in synaptic strength was evident in leptin (116-130), but not leptin (22-56)-treated slices. We have shown that the efficacy of excitatory synaptic transmission is also regulated by leptin in adult hippocampus (Moult et al. 2010). In agreement with this, application of either leptin or leptin (116-130) to adult hippocampal slices resulted in the induction of a persistent increase in synaptic transmission. In contrast, however leptin (22-56) failed to alter excitatory synaptic strength in adult hippocampus.

AMPA receptor trafficking is pivotal for activity-dependent synaptic plasticity (Collingridge et al. 2004) and leptin regulates trafficking of GluA1 to synapses (Moult et al. 2010). In this study, treatment with either leptin or leptin (116-130) increased GluA1 surface expression in cultured hippocampal neurons, whereas leptin (22-56) was without effect. In co-localization studies, the density of GluA1 subunits associated with synapses was increased after application of leptin or leptin (116-130), suggesting that leptin (116-130) parallels the actions of leptin by boosting the synaptic insertion of AMPA receptors. We have shown that leptin-driven trafficking of GluA1 involves inhibition of PTEN (Moult et al. 2010) Similarly in this study, the ability of leptin (116-130) to influence GluA1 trafficking involves inhibition of PTEN, as application of the PTEN inhibitor bpV blocked the increase in GluA1 surface expression induced by leptin (116-130) in hippocampal neurons. These data indicate that like leptin, treatment with leptin (116-130) promotes GluA1 trafficking to hippocampal synapses via inhibition of PTEN. Thus, overall these data indicate that the leptin fragment (116-130) mirrors the actions of leptin as it markedly influences the cellular events underlying learning and memory by regulating AMPA receptor trafficking.

It is known that Aβ inhibits the induction of hippocampal LTP (Shankar et al. 2008), and this detrimental effect of _{Aβ1-42} is reversed by leptin (Doherty et al. 2013). Similarly, leptin (116-130) reversed the acute effects of _{Aβ1-42} in this study as synaptic plasticity was readily induced in hippocampal slices exposed to leptin (116-130) and Aβ₁₋₄₂. Contrastingly, application of leptin (22-56) failed to prevent the detrimental effects of Aβ₁₋₄₂ as no increase in synaptic strength was induced after exposure to Aβ₁₋₄₂ and leptin (22-56). However, in slices exposed to leptin (22-56) post-tetanic potentiation (PTP) and some STP was observed after HFS, suggesting that this fragment may influence the transient enhancement of synaptic strength induced by HFS. As PTP and STP are thought to involve presynaptic expression mechanisms (Zucker and Regehr 2002; Lauri et al. 2007), it is feasible that leptin (22-56) can act pre-synaptically to influence glutamate release mechanisms.

Several studies indicate that Aβ₁₋₄₂ also facilitates the induction of hippocampal LTD (Shankar et al. 2008; Li et al. 2009), and this effect is also reversed by leptin (Doherty et al. 2013). In accordance with these findings, treatment with leptin reduced the magnitude of LTD in Aβ₁₋₄₂-treated slices. Similarly, leptin (116-130), but not leptin (22-56) attenuated the effects of Aβ₁₋₄₂ as the magnitude of LTD was significantly decreased in the presence of leptin (116-130). Moreover, application of either leptin or leptin (116-130) inhibited Aβ₁₋₄₂-driven AMPA receptor removal from hippocampal synapses, whereas treatment with leptin (22-56) was without effect. Thus overall these data demonstrate that leptin (116-130) mirrors the actions of leptin in counteracting the detrimental acute effects of Aβ₁₋₄₂ on hippocampal synaptic function.

Evidence is growing that leptin has neuroprotective actions in various models of neurodegenerative disease. In Parkinson's disease models, treatment with leptin protects dopaminergic neurons from various toxic insults (Weng et al. 2007; Doherty et al. 2008), whereas in AD models of amyloid toxicity, leptin increases neuronal viability via activation of STAT3 and PI3-kinase signalling (Doherty et al. 2008; Guo et al. 2008; Doherty et al. 2013). In this study, leptin and leptin (116-130) enhanced the survival of human neural (SH-SY5Y) cells treated with either Aβ₁₋₄₂ or Cu²⁺. Conversely no change in cell viability was evident after treatment with leptin (22-56), thereby providing further evidence that leptin (116-130) but not leptin (22-56) mirrors the protective actions of leptin.

In these studies, we reveal that signalling via PI3-kinase and STAT3 is essential for leptin (116-130)-mediated neuroprotection as selective inhibition of these pathways eliminated the protective effects of leptin (116-130). Moreover, direct activation of key components of PI3-kinase and STAT3 signalling pathways was observed following administration of leptin (116-130). As stimulation of both PI3-kinase (Doherty et al. 2013; Doherty et al. 2008) and STAT3 signalling cascades (Doherty et al. 2013; Guo et al. 2008) mediate the neuroprotective actions of leptin, these data indicate that leptin (116-130) is activating the same signalling pathways as the full length leptin peptide to induce neuronal survival. This provides further evidence that leptin (116-130) is mirroring the neuronal effects of leptin.

These studies demonstrate that the 116-130 fragment of the leptin molecule enhances hippocampal synaptic plasticity and has neuroprotective effects. As such this fragment is a very interesting therapeutic target to treat memory dysfunction and protect against neurodegeneration in the early stages of AD. To test the functional implications of the effects of leptin 116-130 we examined the effects of acute doses of this fragment on a test of episodic-like memory. This test is particularly appropriate as it models the type of memory that is first compromised in AD. Performance on the task has been shown to be impaired in rodents with damage to the lateral entorhinal cortex (Wilson et al. 2013), the first region to be damaged in AD, and the hippocampus (Langston and Woods, 2010). It has also been shown that the triple transgenic murine model of AD show impaired performance on this task at 6 months of age (Davis et al. 2013). The current data demonstrate powerful cognitive enhancing effects of both leptin and leptin (116-130) as both groups performed significantly better than controls on the OPC task. This is the first time that leptin has been shown to enhance the specific type of memory that degrades in AD and the fact that this cognitive enhancement is also produced by leptin (116-130) suggests that this fragment is a viable tool to treat memory dysfunction caused by damage to the hippocampal-entorhinal network. Recent studies indicate that administration of leptin also protects against Aβ-induced impairments in spatial memory tasks (Tong et al. 2015). Thus, it is feasible that administration of leptin (116-130) will also mirror the effects of leptin and protect against the chronic effects of Aβ on hippocampal-dependent learning and memory.

The current experiments demonstrated enhancement of memory for object-place-context associations. Enhancement of this hippocampal-dependent task is consistent with our findings showing enhancement of hippocampal synaptic plasticity but it remains a possibility that leptin 116-130 may also enhance simpler forms of recognition memory such as object recognition or object-place recognition. These simpler forms of recognition memory are dependent on other areas of the medial temporal lobe network and so future work could examine whether the cognitive enhancement is specific to the hippo-campus or also affects the surrounding cortical inputs. One other consideration is the anxiolytic properties of leptin that have been reported in both normal (Liu et al. 2010) and chronically stressed rats (Lu et al. 2006). Reduced anxiety could potentially affect performance on the spontaneous recognition tasks as less anxious animals may explore more freely. This was not found to be the case in the current study as the levels of exploration in both sample and test phases of the OPC experiment were not different between groups. This is not surprising as animals had extensive handling and pre-training before the OPC test and so levels of anxiety would have been very low in all animals. In conclusion, these data indicate that the leptin (116-130) fragment mirrors the cognitive enhancing effects of leptin as it promotes trafficking of the AMPA receptor subunit GluA1 to synapses, facilitates hippocampal synaptic plasticity and improves performance in an episodic-like memory task. In addition, leptin (116-130) counteracts the detrimental effects of Aβ₁₋₄₂ on hippocampal synaptic function and neuronal viability in various cellular models of amyloid toxicity.

To further refine the precise sequence of leptin (116-130) that is required for its leptin-mimetic effects, hexamer peptides of the molecule were generated by peptide scanning. The potential for these to elicit leptin-like biological effects was tested in vitro.

Two specific leptin hexamers (116-121; 117-122) are effective in mirroring the cognitive enhancing effects of leptin, as treatment of hippocampal slices with either hexamer results in facilitation of hippocampal LTP. In contrast, leptin (124-129) and leptin (125-130) failed to alter the magnitude of LTP suggesting that the N-terminal region of leptin (116-130) is the bioactive region. AMPA receptor trafficking is also critical for hippocampal synaptic plasticity and leptin and leptin (116-130) potently regulate the trafficking of the AMPA receptor subunit, GluA1 (Moult et al, 2010; Malekizadeh et al, 2016). Similarly, exposure of hippocampal neurons to either leptin (116-121) or leptin (117-122) increased the surface expression of GluA1, thereby mirroring the effects of leptin. Contrastingly, treatment with either leptin (124-129) or leptin (125-130) had no effect on GluA1 surface expression in hippocampal neurons.

In accordance with the data above, both leptin (116-121) and leptin (117-122) attenuated Aβ₁₋₄₂-mediated cell death as effectively as either leptin or leptin (116-130). Thus both LDH and MTT assays confirmed that the bioactive region of leptin (116-130) lies in the N-terminal end of the molecule as neither leptin (124-129) nor leptin (125-130) mirrored the neuroprotective effects of leptin or leptin (116-130). Similarly only leptin (116-121) and leptin (117-122) mirrored the leptin or leptin (116-130)-mediated attenuation of p-tau upregulation in response to Aβ₁₋₄₂. Neither leptin (124-129) nor leptin (125-130) had any significant effect.

Taken together the work on the hexamer peptides further refine the sequence required to elicit a leptin-mimetic response, isolating it to the N-terminal of fragment leptin (116-130). Our findings not only reinforce the consensus that the leptin system is an important therapeutic target in AD, but also establish that leptin (116-130), and smaller hexamer fragments of this molecule, may be useful in the development of leptin-mimetic agents for therapeutic use.

### REFERENCES

All references cited herein are hereby incorporated by reference in their entirety.
1. Anderson W W, Collingridge G L. 2007. Capabilities of the WinLTP data acquisition program extending beyond basic LTP experimental functions. J Neurosci Methods. 162: 346-356.
2. Bliss T V, Collingridge G L. 1993. A synaptic model of memory: long-term potentiation in the hippocampus. Nature. 361: 31-39.
3. Clarke R E, Zola S M, Squire L R. 2000. Impaired recognition memory in rats after damage to the hippocampus. J Neurosci. 20: 8853-60.
4. Collingridge G L, Isaac J T, Wang Y T. 2004. Receptor trafficking and synaptic plasticity. Nat Rev Neurosci. 5:952-962.
5. Davis K E, Eacott M J, Easton A, Gigg J. 2013. Episodic-like memory is sensitive to both Alzheimer's-like pathological accumulation and normal ageing processes in mice. Behav Brain Res. 254:73-82.
6. Doherty G H, Beccano-Kelly D, Yan S D, Gunn-Moore F J, Harvey J. 2013. Leptin prevents hippocampal synaptic disruption and neuronal cell death induced by amyloid β. Neurobiol Aging. 34:226-237.
7. Doherty G H, Oldreive C, Harvey J. 2008. Neuroprotective actions of leptin on central and peripheral neurons in vitro. Neuroscience. 154:1297-1307.
8. Eacott M J, Norman G. 2004. Integrated memory for object, place, and context in rats: a possible model of episodic-like memory? J Neurosci. 24:1948-1953.
9. Eikelis N, Wiesner G, Lambert G, Esler M. 2006. Brain leptin resistance in human obesity revisited. Regul Pept. 139:45-51. Farr S A, Banks W A, Morley
10. J E. 2006. Effects of leptin on memory processing. Peptides. 27:1420-1425.
11. Fewlass D C, Noboa K, Pi-Sunyer F X, Johnston J M, Yan S D, Tezapsidis N. 2004. Obesity-related leptin regulates Alzheimer's Abeta. FASEB J. 18:1870-1878.
12. Grasso P, Leinung M C, Ingher S P, Lee D W. 1997. In vivo effects of leptin-related synthetic peptides on body weight and food intake in female ob/ob mice: localization of leptin activity to domains between amino acid residues 106-140. Endocrinology. 138:1413-1418.
13. Grasso P, Rozhayskaya-Arena M, Leinung M C, Lee D W. 2001. [D-LEU-4]-OB3, a synthetic leptin agonist, improves hyperglycemic control in C57BL/6J ob/ob mice. Regul Pept. 101: 123-129.
14. Guo Z, Jiang H, Xu X, Duan W, Mattson M P. 2008. Leptin-mediated cell survival signaling in hippocampal neurons mediated by JAK STAT3 and mitochondrial stabilization. J Biol Chem. 283:1754-1763.
15. Hsieh H, Boehm J, Sato C, Iwatsubo T, Tomita T, Sisodia S, Malinow R. 2006. AMPAR removal underlies Abeta-induced synaptic depression and dendritic spine loss. Neuron. 52: 831-843.
16. Irving A J, Harvey J. 2014. Leptin regulation of hippocampal synaptic function in health and disease. Phil Trans R Soc B. 369: 20130155.
17. Langston R F, Wood E R. 2010. Associative recognition and the hippocampus: differential effects of hippocampal lesions on object-place, object-context and object-place-context memory. Hippocampus. 20:1139-1153.
18. Lauri S E, Palmer M, Segerstrale M, Vesikansa A, Taira T, Collingridge G L. 2007. Presynaptic mechanisms involved in the expression of STP and LTP at CA1 synapses in the hippo-campus. Neuropharmacology. 52:1-11.
19. Li X L, Aou S, Oomura Y, Hori N, Fukunaga K, Hori T. 2002. Impairment of long-term potentiation and spatial memory in leptin receptor-deficient rodents. Neuroscience. 113:607-615.
20. Li S, Hong S, Shepardson N E, Walsh D M, Shankar G M, Selkoe D. 2009. Soluble oligomers of amyloid Beta protein facilitate hippocampal long-term depression by disrupting neuronal glutamate uptake. Neuron. 62:788-801.
21. Lieb W, Beiser A S, Vasan R S, Tan Z S, Au R, Harris T B, Roubenoff R, Auerbach S, DeCarli C, Wolf P A, et al. 2009. Association of plasma leptin levels with incident Alzheimer disease and MRI measures of brain aging. J Am Med Assoc. 302:2565-2572.
22. Liu J, Garza J C, Bronner J, Kim C S, Zhang W, Lu X Y. 2010. Acute administration of leptin produces anxiolytic-like effects: a comparison with fluoxetine. Psychopharmacology. 207 (4): 535-545.
23. Liu S J, Gasperini R, Foa L, Small D H. 2010. Amyloid-beta decreases cell-surface AMPA receptors by increasing intracellular calcium and phosphorylation of GluR2. J Alzheimers Dis. 21:655-666.
24. Luo X, McGregor G, Irving A J, Harvey J. 2015. Leptin induces a novel form of NMDA receptor-dependent LTP at hippocam-pal temporoammonic-CA1 synapses. eNeuro. 2 (3):1-7.
25. Lu X Y, Kim C S, Frazer A, Zhang W. 2006. Leptin: a potential novel antidepressant. Proc Natl Acad Sci USA. 103: 1593-1598.
26. Morash B, Li A, Murphy P R, Wilkinson M, Ur E. 1999. Leptin gene expression in the brain and pituitary gland. Endocrinology. 140:5995-5998.
27. Moult P R, Cross A, Santos S D, Carvalho A L, Lindsay Y, Connolly C N, Irving A J, Leslie N R, Harvey J. 2010. Leptin regulates AMPA receptor trafficking via PTEN inhibition. J Neurosci. 30:4088-4101.
28. Moult P R, Harvey J. 2011. NMDA receptor subunit composition determines the polarity of leptin-induced synaptic plasticity. Neuropharmacology. 61:924-936.
29. Oldreive C E, Doherty G H. 2010. Effects of tumour necrosis factor-alpha on developing cerebellar granule and Purkinje neurons in vitro. J Mol Neurosci. 42:44-52.
30. O'Malley D, MacDonald N, Mizielinska S, Connolly C N, Irving A J, Harvey J. 2007. Leptin promotes rapid dynamic changes in hippocampal dendritic morphology. Mol Cell Neurosci. 35: 559-572.
31. Oomura Y, Hori N, Shiraishi T, Fukunaga K, Takeda H, Tsuji M, Matsumiya T, Ishibashi M, Aou S, Li X L, et al. 2006. Leptin facilitates learning and memory performance and enhances hippocampal CA1 long-term potentiation and CaMK II phosphorylation in rats. Peptides. 27:2738-2749.
32. Power D A, Noel J, Collins R, O'Neill D. 2001. Circulating leptin levels and weight loss in Alzheimer's disease patients. Dement Geriatr Cogn Disord. 12:167-170.
33. Rose G M, Dunwiddie T V. 1986. Induction of hippocampal long-term potentiation using physiologically patterned stimulation. Neurosci Lett. 69:244-248.
34. Rozhayskaya-Arena M, Lee D W, Leinung M C, Grasso P. 2000. Design of a synthetic leptin agonist: effects on energy balance, glucose homeostasis, and thermoregulation. Endocrinology. 141:2501-2507.
35. Samson W K, Murphy T C, Robison D, Vargas T, Tau E, Chang J K. 1996. A 35 amino acid fragment of leptin inhibits feeding in the rat. Endocrinology. 137:5182-5185.
36. Schmid A C, Byrne R D, Vilar R, Woscholski R. 2004. Bisperoxovanadium compounds are potent PTEN inhibitors. FEBS Lett. 566:35-38.
37. Shankar G M, Li S, Mehta T H, Garcia-Munoz A, Shepardson N E, Smith I, Brett F M, Farrell M A, Rowan M J, Lemere C A, et al. 2008. Amyloid-beta protein dimers isolated directly from Alzheimer's brains impair synaptic plasticity and memory. Nat Med. 14:837-842.
38. Shanley L J, Irving A J, Harvey J. 2001. Leptin enhances NMDA receptor function and modulates hippocampal synaptic plasticity. J Neurosci. 21:RC186.
39. Simmons L K, May P C, Tomaselli K J, Rydel R E, Fuson K S, Brigham E F, Wright S, Lieberburg I, Becker G W, Brems D N, et al. 1994. Secondary structure of amyloid beta peptide correlates with neurotoxic activity in vitro. Mol Pharmacol. 45: 373-379.
40. Spiegelman B M, Flier J S. 2001. Obesity and the regulation of energy balance. Cell. 104:531-543.
41. Stranahan A M, Mattson M P. 2012. Metabolic reserve as a determinant of cognitive aging. J Alzheimers Dis. 30:S5-13.
42. Swainson R, Hodges J R, Galton C J, Semple J, Michael A, Dunn B D, Iddon J L, Robbins T W, Sahakian B J. 2001. Early detection and differential diagnosis of Alzheimer's disease and depression with neuropsychological tasks. Dement Geriat Cogn Disord. 12:265-280.
43. Tong J Q, Zhang J, Hao M, Yang J, Han Y F, Liu X J, Shi H, Wu M N, Liu Q S, Qi J S. 2015. Leptin attenuates the detrimental effects of β-amyloid on spatial memory and hippocampal later-phase long term potentiation in rats. Horm Behav. 73: 125-130.
44. Walsh D M, Klyubin I, Fadeeva J V, Cullen W K, Anwyl R, Wolfe M S, Rowan M J, Selkoe D J. 2002. Naturally secreted oligomers of amyloid beta protein potently inhibit hippocampal long-term potentiation in vivo. Nature. 416:535-539.
45. Wayner M J, Armstrong D L, Phelix C F, Oomura Y. 2004. Orexin-A (Hypocretin-1) and leptin enhance LTP in the dentate gyrus of rats in vivo. Peptides. 25:991-996.
46. Weng Z, Signore A P, Gao Y, Wang S, Zhang F, Hastings T, Yin X M, Chen J. 2007. Leptin protects against 6-hydroxydopamine-induced dopaminergic cell death via mitogen-activated protein kinase signaling. J Biol Chem. 282:34479-34491.
47. Wilson D I, Watanabe S, Milner H, Ainge J A. 2013. Lateral entorh-inal cortex is necessary for associative but not non associative recognition memory. Hippocampus. 23:1280-1290.
48. Zucker R S, Regehr W G. 2002. Short-term synaptic plasticity. Annu Rev Physiol. 64:355-405.

1.-34. (canceled)

35. A method of treating a neurological disorder comprising administering to a subject in need, a leptin peptide fragment comprising at least 4 consecutive amino acids located within the region of amino acids 116-125 of leptin.

36. The method according to claim 35, wherein the leptin peptide fragment comprises up to 30 amino acids.

37. The method of claim 35, wherein the leptin peptide fragment comprises one or more amino acids located between amino acids 116-122 of leptin.

38. The method of claim 35, wherein the leptin peptide comprises the sequence:
X1CX2LPX3X4
wherein X1 is selected from G or S; X2 is selected from S, H or P; X3 is selected from Q, H, W, L, P or R and X4 is selected from T, A, or V (SEQ ID NO:14).

39. The method according to claim 35, wherein the leptin peptide comprises amino acids selected from the group consisting of 116-121 of leptin, 117-122 of leptin, 117-125 of leptin, 118-123 of leptin, 119-124 of leptin, 120-125 of leptin, and 116-130 of leptin.

40. The method of claim 35, wherein the leptin peptide fragment comprises a sequence derived from human leptin.

41. The method of claim 35, wherein the leptin peptide fragment has the sequence SCHLPWASGL (SEQ ID NO:22).

42. The method of claim 35, wherein the leptin peptide fragment comprises 1, 2, or 3 deletions, modifications, substitutions and/or additions to the amino acid sequence.

43. The method of claim 35, wherein the leptin peptide fragment is in the form of a cyclic peptide.

44. The method of claim 35, wherein the peptide is in the form of a peptide conjugate, wherein the leptin peptide fragment is conjugated to another peptide, or non-peptide molecule.

45. The method of claim 44, wherein the other peptide or non-peptide molecule is a biologically or pharmaceutically active agent.

46. The method of claim 35, wherein the neurological disorder is a disorder which would benefit from treatment through cognitive enhancement and/or neuroprotection.

47. The method of claim 35, wherein the neurological disorder is selected from the group consisting of age-associated memory impairment or loss, mild cognitive impairment, and Alzheimer's disease.

48. The method of claim 35, wherein the neurological disorder is selected from the group consisting of Parkinson's disease, frontotemporal dementia, progressive supranuclear palsy, Picks disease, corticobasal degeneration, alcoholic dementia, (DLB) dementia with Lewy bodies, Picks' disease, thalamic dementia, hippocampal sclerosis, Hallervorden-Spatz, multiple system atrophy, tauopathies, subacute aterioscleroitic encephalopathy (Binswanger's disease), amyloid angiopathy, vasculitis, prion diseases, and paraneoplastic syndromes.

49. A pharmaceutical formulation comprising a leptin peptide fragment comprising at least 4 consecutive amino acids located within the region of amino acids 116-125 of leptin.

50. The pharmaceutical formulation according to claim 49, wherein the leptin peptide fragment comprises up to 30 amino acids.

51. The pharmaceutical formulation according to claim 49, wherein the leptin peptide fragment comprises one or more amino acids located between amino acids 116-122 of leptin.

52. The pharmaceutical formulation according to claim 49 comprising the sequence:
X1CX2LPX3X4
wherein X1 is selected from G or S; X2 is selected from S, H or P; X3 is selected from Q, H, W, L, P or R and X4 is selected from T, A, or V (SEQ ID NO:14).

53. The pharmaceutical formulation according to claim 49, wherein the leptin peptide fragment comprises amino acids selected from the group consisting of 116-121 of leptin, 117-122 of leptin, 117-125 of leptin, 118-123 of leptin, 119-124 of leptin, 120-125 of leptin, and 116-130 of leptin.

54. The pharmaceutical formulation according to claim 49, wherein the leptin peptide sequence is derived from human leptin.

55. The pharmaceutical formulation according to claim 49, wherein the 116-125 region of leptin has the sequence SCHLPWASGL (SEQ ID NO:22).

56. The pharmaceutical formulation according to claim 49, comprising 1, 2, or 3 deletions, modifications, substitutions and/or additions to the amino acid sequence.

57. The pharmaceutical formulation according to claim 49, wherein the leptin peptide fragment is in the form of a cyclic peptide.

58. The pharmaceutical formulation according to claim 49, wherein the peptide is in the form of a peptide conjugate, wherein the leptin peptide fragment is conjugated to another peptide, or non-peptide molecule.

59. The pharmaceutical formulation according to claim 58, wherein the other peptide or non-peptide molecule is a biologically or pharmaceutically active agent.

60. A cyclic peptide or peptide conjugate comprising a leptin peptide fragment comprising at least 4 consecutive amino acids located within the region of amino acids 116-125 of leptin.

61. The cyclic peptide or peptide conjugate according to claim 60, wherein the leptin peptide fragment comprises up to 30 amino acids.

62. The cyclic peptide or peptide conjugate according to claim 60, wherein the leptin peptide fragment comprises one or more amino acids located between amino acids 116-122 of leptin.

63. The cyclic peptide or peptide conjugate according to claim 60, comprising the sequence:
X1CX2LPX3X4
wherein X1 is selected from G or S; X2 is selected from S, H or P; X3 is selected from Q, H, W, L, P or R and X4 is selected from T, A, or V (SEQ ID NO:14).

64. The cyclic peptide or peptide conjugate according to claim 60, wherein the leptin peptide fragment comprises amino acids selected from the group consisting of 116-121 of leptin, 117-122 of leptin, 117-125 of leptin, 118-123 of leptin, 119-124 of leptin, 120-125 of leptin, and 116-130 of leptin.

65. The cyclic peptide or peptide conjugate according to claims 60, wherein the leptin peptide sequence is derived from human leptin.

66. The cyclic peptide or peptide conjugate according to claim 60, wherein the 116-125 region of leptin has the sequence SCHLPWASGL (SEQ ID NO:22).

67. The cyclic peptide or peptide conjugate according to claims 60, comprising 1, 2, or 3 deletions, modifications, substitutions and/or additions to the amino acid sequence.
